(19) United States
(12) Patent Application Publication
(10) Pub. No.: US 2024/0034814 A1
(43) Pub. Date: Feb. 1, 2024

(54) HALF-LIFE EXTENDING COMPOSITIONS AND METHODS

(71) Applicant: Janux Therapeutics, Inc., San Diego, CA (US)

(72) Inventors: David CAMPBELL, San Diego, CA (US); Thomas R. DIRAIMONDO, San Diego, CA (US); Ramesh BHATT, San Diego, CA (US)

(21) Appl. No.: 18/256,276

(22) PCT Filed: Dec. 7, 2021
(86) PCT No.: PCT/US2021/062238

§ 371 Date: Jun. 7, 2023

Related U.S. Application Data
(60) Provisional application No. 63/122,818, filed on Dec. 8, 2020.

(51) Int. Cl. C07K 16/46

(52) U.S. Cl. CPC C07K 16/468; C07K 2317/565; C07K 2317/569; C07K 2317/24; C07K 2317/622; C07K 2317/92; C07K 2317/55; C07K 2317/31

(57) ABSTRACT

Disclosed herein are polypeptides and polypeptides complexes comprising a half-life extending antibody or antibody fragment. In some embodiments, the half-life extending antibody or antibody fragment is a single domain antibody.

### CROSS-REFERENCE

The present application claims the benefit of U.S. Provisional Application No. 63/122,818, filed Dec. 8, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 30, 2021, is named 52426-731_601_SL.txt and is 39,169 bytes in size.

### SUMMARY

Disclosed herein are isolated polypeptides or polypeptide complexes comprising a half-life extending antibody or antibody fragment (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3. In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3. In some embodiments, the antibody or antibody fragment of H₁ comprises a single domain antibody, a single chain variable fragment, a Fab, or Fab′. In some embodiments, the antibody or antibody fragment of H₁ comprises the single domain antibody. In some embodiments, H₁ comprises an amino acid sequence with at least 90% sequence identity to SEQ ID NO: 4. In some embodiments, H₁ comprises an amino acid sequence with at least 95% sequence identity to SEQ ID NO: 4. In some embodiments, H₁ comprises an amino acid sequence with at least 99% sequence identity to SEQ ID NO: 4. In some embodiments, H₁ comprises an amino acid sequence according to SEQ ID NO: 4. In some embodiments, H₁ comprises an amino acid sequence of at least 110 consecutive amino acid residues of SEQ ID NO: 4. In some embodiments, H₁ comprises an amino acid sequence of at least 120 consecutive amino acid residues of SEQ ID NO: 4. In some embodiments, H₁ comprises an amino acid sequence of at least 110 consecutive amino acid residues of SEQ ID NO: 4 and has at least 95% sequence identity to the at least 110 consecutive amino acid residues of SEQ ID NO: 4. In some embodiments, H₁ comprises an amino acid sequence of at least 120 consecutive amino acid residues of SEQ ID NO: 4 and has at least 95% sequence identity to the at least 120 consecutive amino acid residues of SEQ ID NO: 4. In some embodiments, H₁ is linked to a peptide (P₁) that impairs binding of a first antigen recognizing molecule (A₁) to a first target antigen through a cleavable linker (L₁) that is a substrate for a tumor specific protease in a configuration according to Formula I: A₁-L₁-P₁-H₁. In some embodiments, A₁ is further linked to a second antigen recognizing molecule (A₂). In some embodiments, the polypeptide or polypeptide complex is according to Formula Ia: P₂-L₂-A₂-A₁-L₁-P₁-H₁ wherein P₂ comprises a peptide that impairs binding of A₂ to a second target antigen; and L₂ comprises a second cleavable linker that connects A₂ to P₂ and is a substrate for a tumor specific protease. In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1. In some embodiments, L₃ comprises an amino acid sequence GGGGSGGGS (SEQ ID NO: 19). In some embodiments, A₁ comprises an antibody or antibody fragment. In some embodiments, A₁ comprises an antibody or antibody fragment that is human or humanized. In some embodiments, L₁ is bound to N-terminus of the antibody or antibody fragment of A₁. In some embodiments, A₂ is bound to N-terminus of the antibody or antibody fragment of A₁. In some embodiments, L₁ is bound to the C-terminus of the antibody or antibody fragment of A₁. In some embodiments, A₂ is bound to the C-terminus of the antibody or antibody fragment of A₁. In some embodiments, the antibody or antibody fragment of A₁ comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, the antibody or antibody fragment thereof of A₁ comprises a single chain variable fragment (scFv), a heavy chain variable domain (VH domain), a light chain variable domain (VL domain), or a variable domain (VHH) of a camelid derived single domain antibody. In some embodiments, the first target antigen comprises an effector cell antigen. In some embodiments, the first target antigen is CD3. In some embodiments, A₁ comprises an anti-CD3e single chain variable fragment. In some embodiments, A₁ comprises an anti-CD3e single chain variable fragment that has a KD binding of 1 μM or less to CD3 on CD3 expressing cells. In some embodiments, A₁ comprises a variable light chain and variable heavy chain each of which is capable of specifically binding to human CD3. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, wherein the polypeptide or polypeptide complex of formula I binds to an effector cell when L₁ is cleaved by the tumor specific protease. In some embodiments, the polypeptide or polypeptide complex of formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε. In some embodiments, A₂ comprises an antibody or antibody fragment. In some embodiments, the antibody or antibody fragment thereof of A₂ comprises a single chain variable fragment, a single domain antibody, Fab′, or a Fab. In some embodiments, the antibody or antibody fragment thereof of A₂ comprises a single chain variable fragment (scFv), a heavy chain variable domain (VH domain), a light chain variable domain (VL domain), or a variable domain (VHH) of a camelid derived single domain antibody. In some embodiments, the antibody or antibody fragment thereof of A₂ is humanized or human. In some embodiments, A₂ is the Fab or Fab′. In some embodiments, the Fab or Fab′ comprises (a) a Fab light chain polypeptide and (b) a Fab heavy chain polypeptide. In some embodiments, the second target antigen comprises a tumor antigen. In some embodiments, the antibody or antibody fragment of A₂ comprises an epidermal growth factor receptor (EGFR) binding domain. In some embodiments, the antibody or antibody fragment of A₂ comprises a mesothelin binding domain. In some embodiments, P₁ impairs binding of A₁ to the first target antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ has less than 70% sequence homology to the first target antigen. In some embodiments, P₂ impairs binding of A₂ to the second target antigen. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ has less than 70% sequence homology to second target antigen. In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino acids in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide. In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ has a formula comprising (G₂S)ₙ, wherein n is an integer from 1 to 3 (SEQ ID NO: 20). In some embodiments, L₁ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1. In some embodiments, P₁ becomes unbound from A₁ when L₁ is cleaved by the tumor specific protease thereby exposing A₁ to the first target antigen. In some embodiments, P₂ becomes unbound from A₂ when L₂ is cleaved by the tumor specific protease thereby exposing A₂ to the second target antigen. In some embodiments, the tumor specific protease is selected from the group consisting of metalloprotease, serine protease, cysteine protease, threonine protease, and aspartic protease. In some embodiments, L₁ or L₂ comprises a urokinase cleavable amino acid sequence, a matriptase cleavable amino acid sequence, matrix metalloprotease cleavable amino acid sequence, or a legumain cleavable amino acid sequence. In some embodiments, L₁ or L₂ comprises an amino acid sequence selected from the group consisting of GGGGSLSGRSDNHGSSGT (SEQ ID NO: 21), GGGGSSGGSGGSGLSGRSDNHGSSGT (SEQ ID NO: 22), ASGRSDNH (SEQ ID NO: 23), LAGRSDNH (SEQ ID NO: 24), ISSGLASGRSDNH (SEQ ID NO: 25), ISSGLLAGRSDNH (SEQ ID NO: 26), LSGRSDNH (SEQ ID NO: 27), ISSGLLSGRSDNP (SEQ ID NO: 28), ISSGLLSGRSDNH (SEQ ID NO: 29), LSGRSDNHSPLGLAGS (SEQ ID NO: 30), SPLGLAGSLSGRSDNH (SEQ ID NO: 31), SPLGLSGRSDNH (SEQ ID NO: 32), LAGRSDNHSPLGLAGS (SEQ ID NO: 33), LSGRSDNHVPLSLKMG (SEQ ID NO: 34), and LSGRSDNHVPLSLSMG (SEQ ID NO: 35). In some embodiments, L₁ or L₂ comprises an amino acid sequence selected from the group consisting of ASGRSDNH (SEQ ID NO: 23), LAGRSDNH (SEQ ID NO: 24), ISSGLASGRSDNH (SEQ ID NO: 25), and ISSGLLAGRSDNH (SEQ ID NO: 26). In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

Disclosed herein are pharmaceutical compositions comprising: (i) the polypeptides or polypeptide complexes of any one of the previous embodiments; and (ii) a pharmaceutically acceptable excipient.

Disclosed herein are isolated recombinant nucleic acid molecules encoding the polypeptides or polypeptide complexes of any one of the previous embodiments.

Disclosed herein are isolated polypeptides or polypeptide complexes according to Formula II: L₁ₐ-P₁ₐ-H₁ₐ wherein: L₁ₐ comprises a tumor specific protease-cleaved linking moiety that when uncleaved connects P₁ₐ to a first antigen recognizing molecule that binds to a first target antigen; P₁ₐ comprises a peptide that impairs binding of the first antigen recognizing molecule to the first target antigen when L₁ₐ is uncleaved; and H₁ₐ comprises a half-life extending molecule that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3. In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3. In some embodiments, the antibody or antibody fragment of H₁ₐ comprises a single domain antibody, a single chain variable fragment, a Fab, or Fab′. In some embodiments, the antibody or antibody fragment of H₁ comprises the single domain antibody. In some embodiments, H₁ₐ comprises an amino acid sequence with at least 90% sequence identity to SEQ ID NO: 4. In some embodiments, H₁ₐ comprises an amino acid sequence with at least 95% sequence identity to SEQ ID NO: 4. In some embodiments, H₁ₐ comprises an amino acid sequence with at least 99% sequence identity to SEQ ID NO: 4. In some embodiments, H₁ₐ comprises an amino acid sequence according to SEQ ID NO: 4. In some embodiments, H₁ₐ comprises an amino acid sequence of at least 110 consecutive amino acid residues of SEQ ID NO: 4. In some embodiments, H₁ₐ comprises an amino acid sequence of at least 120 consecutive amino acid residues of SEQ ID NO: 4. In some embodiments, H₁ₐ comprises an amino acid sequence of at least 110 consecutive amino acid residues of SEQ ID NO: 4 and has at least 95% sequence identity to the at least 110 consecutive amino acid residues of SEQ ID NO: 4. In some embodiments, H₁ₐ comprises an amino acid sequence of at least 120 consecutive amino acid residues of SEQ ID NO: 4 and has at least 95% sequence identity to the at least 120 consecutive amino acid residues of SEQ ID NO: 4. In some embodiments, P₁ₐ when L₁ₐ is uncleaved impairs binding of the first antigen recognizing molecule to the effector cell antigen. In some embodiments, the first antigen recognizing molecule comprises an antibody or antibody fragment. In some embodiments, the effector cell antigen is an anti-CD3 effector cell antigen. In some embodiments, P₁ₐ has less than 70% sequence homology to the effector cell antigen. In some embodiments, P₁ₐ comprises a peptide sequence of at least 10 amino acids in length. In some embodiments, P₁ₐ comprises a peptide sequence of at least 10 amino acids in length and no more than 20 amino acids in length. In some embodiments, P₁ₐ comprises a peptide sequence of at least 16 amino acids in length. In some embodiments, P₁ₐ comprises a peptide sequence of no more than 40 amino acids in length. In some embodiments, P₁ₐ comprises at least two cysteine amino acid residues. In some embodiments, P₁ₐ comprises a cyclic peptide or a linear peptide. In some embodiments, P₁ₐ comprises a cyclic peptide. In some embodiments, P₁ₐ comprises a linear peptide. In some embodiments, H₁ₐ comprises a linking moiety (L₃ₐ) that connects H₁ₐ to P₁ₐ. In some embodiments, L₃ₐ is a peptide sequence having at least 5 to no more than 50 amino acids. In some embodiments, L₃ₐ is a peptide sequence having at least 10 to no more than 30 amino acids. In some embodiments, L₃ₐ is a peptide sequence having at least 10 amino acids. In some embodiments, L₃ₐ is a peptide sequence having at least 18 amino acids. In some embodiments, L₃ₐ is a peptide sequence having at least 26 amino acids. In some embodiments, L₃ₐ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1. In some embodiments, L₃ₐ comprises an amino acid sequence according to GGGGSGGGS (SEQ ID NO: 19).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a binding curve of a single domain antibody to bovine serum albumin.

FIG. 1B illustrates a binding curve of a single domain antibody to mouse serum albumin.

FIG. 1C illustrates a binding curve of a single domain antibody to cynomolgus monkey serum albumin.

FIG. 1D illustrates a binding curve of a single domain antibody to human serum albumin.

FIG. 2A illustrates titration data of HE-1 anti-albumin at p.H 7.4.

FIG. 2B illustrates titration data of HE-1 anti-albumin at p.H 5.5.

FIG. 3A illustrates titration data of single domain antibody binding to cynomolgus monkey serum albumin at pH 7.4.

FIG. 3B illustrates titration data of single domain antibody binding to human serum albumin at pH 7.4.

FIG. 3C illustrates titration data of single domain antibody binding to cynomolgus monkey serum albumin at pH 5.5.

FIG. 3D illustrates titration data of single domain antibody binding to human serum albumin at pH 5.5.

FIG. 4 illustrates binding data of a single domain antibody to human serum albumin without blocking FcRn recognition.

FIG. 5 illustrates binding data of polypeptide complexes PC1 and PC2 to PSMA as measured by ELISA.

FIG. 6 illustrates binding data of polypeptide complexes PC1 and PC2 to CD3 as measured by ELISA.

FIG. 7 illustrates killing of 22Rv1 tumor cells mediated by polypeptide complexes PC1 and PC2 in the presence of CD8+ T cells.

FIG. 8 illustrates pharmacokinetic data of polypeptide complex PC1 in cynomolgus monkeys after a single IV bolus injection.

FIG. 9 illustrates pharmacokinetic data of polypeptide complex PC2 in cynomolgus monkeys after a single IV bolus injection.

FIG. 10 illustrates binding data of polypeptide complexes PC1 and PC4 to TROP2 as measured by ELISA.

FIG. 11 illustrates binding data of polypeptide complexes PC1 and PC4 binding to CD3 as measured by ELISA.

FIG. 12 illustrates killing of H292 tumor cells mediated by polypeptide complexes PC3 and PC4 in the presence of CD8+ T cells.

FIG. 13 illustrates pharmacokinetic data of polypeptide complex PC3 in cynomolgus monkeys after a single IV bolus injection.

FIG. 14 illustrates pharmacokinetic data of polypeptide complex PC4 in cynomolgus monkeys after a single IV bolus injection.

### DETAILED DESCRIPTION

Protein-based therapies such as antibodies and bispecific or multispecific antibodies, such as T cell engagers, have proven effective for a variety diseases and disorders. As with any therapy, there is a need to minimize off-target effects of the protein-based therapy in healthy tissue while maintaining activity of the protein-based therapy in disease tissue. One such strategy is to create an inactive form of the protein-based therapy in which a necessary binding site on the protein-based therapy is blocked with a peptide linked to the protein-based therapy, thereby preventing the protein-based therapy from binding or interacting with its target antigen when in healthy tissue. For activating the protein-based therapy in the desired disease-state microenvironment, the peptide is linked to the protein-based therapy with a linker that is cleavable by a protease that is specific to the disease-state microenvironment. The peptide is then released from the protein-based therapy when in the disease-state microenvironment.

In order to improve pharmacokinetic properties of the protein-based therapies, a half-life extending molecule is attached to the therapy. One such location for attaching the half-life extending molecule to the protein-based therapy is to the peptide that blocks the protein-based therapy from binding or interacting with its target antigen when in healthy tissue. When the peptide is cleaved in the disease-state microenvironment, the half-life extending molecule is also released from the protein-based therapy.

Disclosed herein are isolated polypeptides or polypeptide complexes comprising half-life extending antibodies or antibody fragments (H₁) that comprise complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3.

In some embodiments, the isolated polypeptides or polypeptide complexes comprising half-life extending antibodies or antibody fragments (H₁) are used in a method of treating cancer in a subject in need thereof. In some embodiments, is a method of treating cancer comprising administering to a subject in need thereof an isolated polypeptide or polypeptide complex comprising half-life extending antibody or antibody fragments (H₁) that comprise complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3. In some embodiments, the cancer has cells that express EGFR. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least two of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least three of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise 1 amino acid modification in at least one of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise 1 amino acid modification in at least two of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise 1 amino acid modification in at least three of the HC-CDR1, HC-CDR2, or HC-CDR3.

In some embodiments, H₁ comprises an amino acid sequence with at least 70% sequence identity to SEQ ID NO: 4. In some embodiments, H₁ comprises an amino acid sequence with at least 80% sequence identity to SEQ ID NO: 4. In some embodiments, H₁ comprises an amino acid sequence with at least 85% sequence identity to SEQ ID NO: 4. In some embodiments, H₁ comprises an amino acid sequence with at least 90% sequence identity to SEQ ID NO: 4. In some embodiments, H₁ comprises an amino acid sequence with at least 91% sequence identity to SEQ ID NO: 4. In some embodiments, H₁ comprises an amino acid sequence with at least 92% sequence identity to SEQ ID NO: 4. In some embodiments, H₁ comprises an amino acid sequence with at least 93% sequence identity to SEQ ID NO: 4. In some embodiments, H₁ comprises an amino acid sequence with at least 94% sequence identity to SEQ ID NO: 4. In some embodiments, H₁ comprises an amino acid sequence with at least 95% sequence identity to SEQ ID NO: 4. In some embodiments, H₁ comprises an amino acid sequence with at least 96% sequence identity to SEQ ID NO: 4. In some embodiments, H₁ comprises an amino acid sequence with at least 97% sequence identity to SEQ ID NO: 4. In some embodiments, H₁ comprises an amino acid sequence with at least 98% sequence identity to SEQ ID NO: 4. In some embodiments, H₁ comprises an amino acid sequence with at least 99% sequence identity to SEQ ID NO: 4. In some embodiments, H₁ comprises an amino acid sequence according to SEQ ID NO: 4.

In some embodiments, H₁ comprises an amino acid sequence of at least 110 consecutive amino acid residues of SEQ ID NO: 4. In some embodiments, H₁ comprises an amino acid sequence of at least 115 consecutive amino acid residues of SEQ ID NO: 4. In some embodiments, H₁ comprises an amino acid sequence of at least 120 consecutive amino acid residues of SEQ ID NO: 4.

In some embodiments, H₁ comprises an amino acid sequence of at least 110 consecutive amino acid residues of SEQ ID NO: 4 and has at least 90% sequence identity to the at least 110 consecutive amino acid residues of SEQ ID NO: 4. In some embodiments, H₁ comprises an amino acid sequence of at least 110 consecutive amino acid residues of SEQ ID NO: 4 and has at least 95% sequence identity to the at least 110 consecutive amino acid residues of SEQ ID NO: 4. In some embodiments, H₁ comprises an amino acid sequence of at least 110 consecutive amino acid residues of SEQ ID NO: 4 and has at least 99% sequence identity to the at least 110 consecutive amino acid residues of SEQ ID NO: 4.

In some embodiments, H₁ comprises an amino acid sequence of at least 115 consecutive amino acid residues of SEQ ID NO: 4 and has at least 90% sequence identity to the at least 115 consecutive amino acid residues of SEQ ID NO: 4. In some embodiments, H₁ comprises an amino acid sequence of at least 115 consecutive amino acid residues of SEQ ID NO: 4 and has at least 95% sequence identity to the at least 115 consecutive amino acid residues of SEQ ID NO: 4. In some embodiments, H₁ comprises an amino acid sequence of at least 115 consecutive amino acid residues of SEQ ID NO: 4 and has at least 99% sequence identity to the at least 115 consecutive amino acid residues of SEQ ID NO: 4.

In some embodiments, H₁ comprises an amino acid sequence of at least 120 consecutive amino acid residues of SEQ ID NO: 4 and has at least 90% sequence identity to the at least 120 consecutive amino acid residues of SEQ ID NO: 4. In some embodiments, H₁ comprises an amino acid sequence of at least 120 consecutive amino acid residues of SEQ ID NO: 4 and has at least 95% sequence identity to the at least 120 consecutive amino acid residues of SEQ ID NO: 4. In some embodiments, H₁ comprises an amino acid sequence of at least 120 consecutive amino acid residues of SEQ ID NO: 4 and has at least 99% sequence identity to the at least 120 consecutive amino acid residues of SEQ ID NO: 4.

Disclosed herein are isolated polypeptides or polypeptide complexes comprising half-life extending antibodies or antibody fragments (H₁) that comprise complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least one of the HC-CDR1, HC-CDR2, or HC-CDR3.

In some embodiments, the isolated polypeptides or polypeptide complexes comprising half-life extending antibodies or antibody fragments (H₁) are used in a method of treating cancer in a subject in need thereof. In some embodiments, is a method of treating cancer comprising administering to a subject in need thereof an isolated polypeptide or polypeptide complex comprising half-life extending antibody or antibody fragments (H₁) that comprise complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least one of the HC-CDR1, HC-CDR2, or HC-CDR3. In some embodiments, the cancer has cells that express EGFR. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least two of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least three of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise 1 amino acid modification in at least one of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise 1 amino acid modification in at least two of the HC-CDR1, HC-CDR2, or HC-CDR3.

In some embodiments, H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise 1 amino acid modification in at least three of the HC-CDR1, HC-CDR2, or HC-CDR3.

In some embodiments, H₁ comprises an amino acid sequence with at least 70% sequence identity to SEQ ID NO: 6. In some embodiments, H₁ comprises an amino acid sequence with at least 80% sequence identity to SEQ ID NO: 6. In some embodiments, H₁ comprises an amino acid sequence with at least 85% sequence identity to SEQ ID NO: 6. In some embodiments, H₁ comprises an amino acid sequence with at least 90% sequence identity to SEQ ID NO: 6. In some embodiments, H₁ comprises an amino acid sequence with at least 91% sequence identity to SEQ ID NO: 6. In some embodiments, H₁ comprises an amino acid sequence with at least 92% sequence identity to SEQ ID NO: 6. In some embodiments, H₁ comprises an amino acid sequence with at least 93% sequence identity to SEQ ID NO: 6. In some embodiments, H₁ comprises an amino acid sequence with at least 94% sequence identity to SEQ ID NO: 6. In some embodiments, H₁ comprises an amino acid sequence with at least 95% sequence identity to SEQ ID NO: 6. In some embodiments, H₁ comprises an amino acid sequence with at least 96% sequence identity to SEQ ID NO: 6. In some embodiments, H₁ comprises an amino acid sequence with at least 97% sequence identity to SEQ ID NO: 6. In some embodiments, H₁ comprises an amino acid sequence with at least 98% sequence identity to SEQ ID NO: 6. In some embodiments, H₁ comprises an amino acid sequence with at least 99% sequence identity to SEQ ID NO: 6. In some embodiments, H₁ comprises an amino acid sequence according to SEQ ID NO: 6.

In some embodiments, H₁ comprises an amino acid sequence of at least 110 consecutive amino acid residues of SEQ ID NO: 6. In some embodiments, H₁ comprises an amino acid sequence of at least 115 consecutive amino acid residues of SEQ ID NO: 6. In some embodiments, H₁ comprises an amino acid sequence of at least 120 consecutive amino acid residues of SEQ ID NO: 6.

In some embodiments, H₁ comprises an amino acid sequence of at least 110 consecutive amino acid residues of SEQ ID NO: 6 and has at least 90% sequence identity to the at least 110 consecutive amino acid residues of SEQ ID NO: 6. In some embodiments, H₁ comprises an amino acid sequence of at least 110 consecutive amino acid residues of SEQ ID NO: 6 and has at least 95% sequence identity to the at least 110 consecutive amino acid residues of SEQ ID NO: 6. In some embodiments, H₁ comprises an amino acid sequence of at least 110 consecutive amino acid residues of SEQ ID NO: 6 and has at least 99% sequence identity to the at least 110 consecutive amino acid residues of SEQ ID NO: 6.

In some embodiments, H₁ comprises an amino acid sequence of at least 115 consecutive amino acid residues of SEQ ID NO: 6 and has at least 90% sequence identity to the at least 115 consecutive amino acid residues of SEQ ID NO: 6. In some embodiments, H₁ comprises an amino acid sequence of at least 115 consecutive amino acid residues of SEQ ID NO: 6 and has at least 95% sequence identity to the at least 115 consecutive amino acid residues of SEQ ID NO: 6. In some embodiments, H₁ comprises an amino acid sequence of at least 115 consecutive amino acid residues of SEQ ID NO: 6 and has at least 99% sequence identity to the at least 115 consecutive amino acid residues of SEQ ID NO: 6.

In some embodiments, H₁ comprises an amino acid sequence of at least 120 consecutive amino acid residues of SEQ ID NO: 6 and has at least 90% sequence identity to the at least 120 consecutive amino acid residues of SEQ ID NO: 6. In some embodiments, H₁ comprises an amino acid sequence of at least 120 consecutive amino acid residues of SEQ ID NO: 6 and has at least 95% sequence identity to the at least 120 consecutive amino acid residues of SEQ ID NO: 6. In some embodiments, H₁ comprises an amino acid sequence of at least 120 consecutive amino acid residues of SEQ ID NO: 6 and has at least 99% sequence identity to the at least 120 consecutive amino acid residues of SEQ ID NO: 6.

In some embodiments, the antibody or antibody fragment of H₁ comprises a single domain antibody, a single chain variable fragment, a Fab, or Fab′. In some embodiments, the antibody or antibody fragment of H₁ comprises a single domain antibody. In some embodiments, the antibody or antibody fragment of H₁ comprises a Fab In some embodiments, the antibody or antibody fragment of H₁ comprises a Fab′. In some embodiments, the antibody or antibody fragment of H₁ comprises a scFv.

In some embodiments, the H₁ binds to serum albumin selected from human serum albumin, cynomolgus serum albumin, and mouse serum albumin. In some embodiments, H₁ binds to human serum albumin and cynomolgus serum albumin with comparable binding affinity (Kd). In some embodiments, H₁ binds to mouse serum albumin with a binding affinity (Kd) that is about 1.5 fold to about 20 fold weaker than the binding affinity (Kd) of said protein towards human and cynomolgus serum albumin. In some embodiments, H₁ binds to human serum albumin with a human Kd (hKd) between about 1 nM and about 100 nM and to cynomolgus serum albumin with a cynomolgus Kd (cKd) between 1 nM and 100 nM. In some embodiments, the hKd and the cKd of H₁ are between 1 nM and about 5 nM, or about 5 nM and about 10 nM. In some embodiments, the hKd and cKd of H₁ are between about 1 nM and about 2 nM, about 2 nM and about 3 nM, about 3 nM and about 4 nM, about 4 nM and about 5 nM, about 5 nM and about 6 nM, about 6 nM and about 7 nM, about 7 nM and about 8 nM, about 8 nM and about 9 nM, or about 9 nM and about 10 nM. In some embodiments, the ratio between the hKd and cKd (hKd:cKd) of H₁ ranges from about 20:1 to about 1:2.

In some embodiments, H₁ protein comprises an elimination half-time of at least 12 hours, at least 20 hours, at least 25 hours, at least 30 hours, at least 35 hours, at least 40 hours, at least 45 hours, at least 50 hours, or at least 100 hours.

In some embodiments, the antibody or antibody fragment of H₁ comprises a heavy chain variable domain. In some embodiments, the heavy chain variable domain of H₁ comprises a variable domain of an IgG1, IgG2, IgG3, or IgG4 heavy chain. In some embodiments, the antibody or antibody fragment of H₁ comprises a light chain variable domain. In some embodiments, the light chain variable domain of H₁ comprises a variable domain of an IgG1, IgG2, IgG3, or IgG4 light chain.

In some embodiments, H₁ is linked to a peptide (P₁) that impairs binding of a first antigen recognizing molecule (A₁) to a first target antigen through a cleavable linker (L₁) that is a substrate for a tumor specific protease in a configuration according to Formula I:

A₁-L₁-P₁-H₁ (Formula I).

In some embodiments, A₁ is further linked to a second antigen recognizing molecule (A₂). In some embodiments, the polypeptide or polypeptide complex is according to Formula Ia:

P₂-L₂-A₂-A₁-L₁-P₁-H₁ (Formula Ia)

wherein P₂ comprises a peptide that impairs binding of A₂ to a second target antigen; and L₂ comprises a second cleavable linker that connects A₂ to P₂ and is a substrate for a tumor specific protease.

Disclosed herein, in some embodiments, are polypeptides or polypeptide complexes according to Formula II:

L₁ₐ-P₁ₐ-H₁ₐ (Formula II)

wherein: L₁ₐ comprises a tumor specific protease-cleaved linking moiety that when uncleaved connects P₁ₐ to a first antigen recognizing molecule that binds to a first target antigen; P₁ₐ comprises a peptide that impairs binding of the first antigen recognizing molecule to the first target antigen when L₁ₐ is uncleaved; and H₁ₐ comprises a half-life extending molecule that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least two of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least three of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3; and wherein the CDRs comprise 1 amino acid modification in at least one of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3; and wherein the CDRs comprise 1 amino acid modification in at least two of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3; and wherein the CDRs comprise 1 amino acid modification in at least three of the HC-CDR1, HC-CDR2, or HC-CDR3.

In some embodiments, H₁ₐ comprises an amino acid sequence with at least 70% sequence identity to SEQ ID NO: 4. In some embodiments, H₁ₐ comprises an amino acid sequence with at least 80% sequence identity to SEQ ID NO: 4. In some embodiments, H₁ₐ comprises an amino acid sequence with at least 85% sequence identity to SEQ ID NO: 4. In some embodiments, H₁ₐ comprises an amino acid sequence with at least 90% sequence identity to SEQ ID NO: 4. In some embodiments, H₁ₐ comprises an amino acid sequence with at least 91% sequence identity to SEQ ID NO: 4. In some embodiments, H₁ₐ comprises an amino acid sequence with at least 92% sequence identity to SEQ ID NO: 4. In some embodiments, H₁ₐ comprises an amino acid sequence with at least 93% sequence identity to SEQ ID NO: 4. In some embodiments, H₁ₐ comprises an amino acid sequence with at least 94% sequence identity to SEQ ID NO: 4. In some embodiments, H₁ₐ comprises an amino acid sequence with at least 95% sequence identity to SEQ ID NO: 4. In some embodiments, H₁ₐ comprises an amino acid sequence with at least 96% sequence identity to SEQ ID NO: 4. In some embodiments, H₁ₐ comprises an amino acid sequence with at least 97% sequence identity to SEQ ID NO: 4. In some embodiments, H₁ₐ comprises an amino acid sequence with at least 98% sequence identity to SEQ ID NO: 4. In some embodiments, H₁ₐ comprises an amino acid sequence with at least 99% sequence identity to SEQ ID NO: 4. In some embodiments, H₁ₐ comprises an amino acid sequence according to SEQ ID NO: 4.

In some embodiments, H₁ₐ comprises an amino acid sequence of at least 110 consecutive amino acid residues of SEQ ID NO: 4. In some embodiments, H₁ₐ comprises an amino acid sequence of at least 115 consecutive amino acid residues of SEQ ID NO: 4. In some embodiments, H₁ₐ comprises an amino acid sequence of at least 120 consecutive amino acid residues of SEQ ID NO: 4.

In some embodiments, H₁ₐ comprises an amino acid sequence of at least 110 consecutive amino acid residues of SEQ ID NO: 4 and has at least 90% sequence identity to the at least 110 consecutive amino acid residues of SEQ ID NO: 4. In some embodiments, H₁ₐ comprises an amino acid sequence of at least 110 consecutive amino acid residues of SEQ ID NO: 4 and has at least 95% sequence identity to the at least 110 consecutive amino acid residues of SEQ ID NO: 4. In some embodiments, H₁ₐ comprises an amino acid sequence of at least 110 consecutive amino acid residues of SEQ ID NO: 4 and has at least 99% sequence identity to the at least 110 consecutive amino acid residues of SEQ ID NO: 4.

In some embodiments, H₁ₐ comprises an amino acid sequence of at least 115 consecutive amino acid residues of SEQ ID NO: 4 and has at least 90% sequence identity to the at least 115 consecutive amino acid residues of SEQ ID NO: 4. In some embodiments, H₁ₐ comprises an amino acid sequence of at least 115 consecutive amino acid residues of SEQ ID NO: 4 and has at least 95% sequence identity to the at least 115 consecutive amino acid residues of SEQ ID NO: 4. In some embodiments, H₁ₐ comprises an amino acid sequence of at least 115 consecutive amino acid residues of SEQ ID NO: 4 and has at least 99% sequence identity to the at least 115 consecutive amino acid residues of SEQ ID NO: 4.

In some embodiments, H₁ₐ comprises an amino acid sequence of at least 120 consecutive amino acid residues of SEQ ID NO: 4 and has at least 90% sequence identity to the at least 120 consecutive amino acid residues of SEQ ID NO: 4. In some embodiments, H₁ₐ comprises an amino acid sequence of at least 120 consecutive amino acid residues of SEQ ID NO: 4 and has at least 95% sequence identity to the at least 120 consecutive amino acid residues of SEQ ID NO: 4. In some embodiments, H₁ₐ comprises an amino acid sequence of at least 120 consecutive amino acid residues of SEQ ID NO: 4 and has at least 99% sequence identity to the at least 120 consecutive amino acid residues of SEQ ID NO: 4.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least two of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least three of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 5; and wherein the CDRs comprise 1 amino acid modification in at least one of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 5; and wherein the CDRs comprise 1 amino acid modification in at least two of the HC-CDR1, HC-CDR2, or HC-CDR3.

In some embodiments, H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 5; and wherein the CDRs comprise 1 amino acid modification in at least three of the HC-CDR1, HC-CDR2, or HC-CDR3.

In some embodiments, H₁ₐ comprises an amino acid sequence with at least 70% sequence identity to SEQ ID NO: 6. In some embodiments, H₁ₐ comprises an amino acid sequence with at least 80% sequence identity to SEQ ID NO: 6. In some embodiments, H₁ₐ comprises an amino acid sequence with at least 85% sequence identity to SEQ ID NO: 6. In some embodiments, H₁ₐ comprises an amino acid sequence with at least 90% sequence identity to SEQ ID NO: 6. In some embodiments, H₁ₐ comprises an amino acid sequence with at least 91% sequence identity to SEQ ID NO: 6. In some embodiments, H₁ₐ comprises an amino acid sequence with at least 92% sequence identity to SEQ ID NO: 6. In some embodiments, H₁ₐ comprises an amino acid sequence with at least 93% sequence identity to SEQ ID NO: 6. In some embodiments, H₁ₐ comprises an amino acid sequence with at least 94% sequence identity to SEQ ID NO: 6.

In some embodiments, H₁ₐ comprises an amino acid sequence with at least 95% sequence identity to SEQ ID NO: 6. In some embodiments, H₁ₐ comprises an amino acid sequence with at least 96% sequence identity to SEQ ID NO: 6. In some embodiments, H₁ₐ comprises an amino acid sequence with at least 97% sequence identity to SEQ ID NO: 6. In some embodiments, H₁ₐ comprises an amino acid sequence with at least 98% sequence identity to SEQ ID NO: 6. In some embodiments, H₁ₐ comprises an amino acid sequence with at least 99% sequence identity to SEQ ID NO: 6. In some embodiments, H₁ₐ comprises an amino acid sequence according to SEQ ID NO: 6.

In some embodiments, H₁ₐ comprises an amino acid sequence of at least 110 consecutive amino acid residues of SEQ ID NO: 6. In some embodiments, H₁ₐ comprises an amino acid sequence of at least 115 consecutive amino acid residues of SEQ ID NO: 6. In some embodiments, H₁ₐ comprises an amino acid sequence of at least 120 consecutive amino acid residues of SEQ ID NO: 6.

In some embodiments, H₁ₐ comprises an amino acid sequence of at least 110 consecutive amino acid residues of SEQ ID NO: 6 and has at least 90% sequence identity to the at least 110 consecutive amino acid residues of SEQ ID NO: 6. In some embodiments, H₁ₐ comprises an amino acid sequence of at least 110 consecutive amino acid residues of SEQ ID NO: 6 and has at least 95% sequence identity to the at least 110 consecutive amino acid residues of SEQ ID NO: 6. In some embodiments, H₁ₐ comprises an amino acid sequence of at least 110 consecutive amino acid residues of SEQ ID NO: 6 and has at least 99% sequence identity to the at least 110 consecutive amino acid residues of SEQ ID NO: 6.

In some embodiments, H₁ₐ comprises an amino acid sequence of at least 115 consecutive amino acid residues of SEQ ID NO: 6 and has at least 90% sequence identity to the at least 115 consecutive amino acid residues of SEQ ID NO: 6. In some embodiments, H₁ₐ comprises an amino acid sequence of at least 115 consecutive amino acid residues of SEQ ID NO: 6 and has at least 95% sequence identity to the at least 115 consecutive amino acid residues of SEQ ID NO: 6. In some embodiments, H₁ₐ comprises an amino acid sequence of at least 115 consecutive amino acid residues of SEQ ID NO: 6 and has at least 99% sequence identity to the at least 115 consecutive amino acid residues of SEQ ID NO: 6.

In some embodiments, H₁ₐ comprises an amino acid sequence of at least 120 consecutive amino acid residues of SEQ ID NO: 6 and has at least 90% sequence identity to the at least 120 consecutive amino acid residues of SEQ ID NO: 6. In some embodiments, H₁ₐ comprises an amino acid sequence of at least 120 consecutive amino acid residues of SEQ ID NO: 6 and has at least 95% sequence identity to the at least 120 consecutive amino acid residues of SEQ ID NO: 6. In some embodiments, H₁ₐ comprises an amino acid sequence of at least 120 consecutive amino acid residues of SEQ ID NO: 6 and has at least 99% sequence identity to the at least 120 consecutive amino acid residues of SEQ ID NO: 6.

In some embodiments, the antibody or antibody fragment of H₁ₐ comprises a single domain antibody, a single chain variable fragment, a Fab, or Fab′. In some embodiments, the antibody or antibody fragment of H₁ₐ comprises a single domain antibody. In some embodiments, the antibody or antibody fragment of H₁ₐ comprises a Fab. In some embodiments, the antibody or antibody fragment of H₁ₐ comprises a Fab′. In some embodiments, the antibody or antibody fragment of H₁ₐ comprises a scFv.

In some embodiments, the antibody or antibody fragment of H₁ₐ comprises a heavy chain variable domain. In some embodiments, the heavy chain variable domain of H₁ₐ comprises a variable domain of an IgG1, IgG2, IgG3, or IgG4 heavy chain. In some embodiments, the antibody or antibody fragment of H₁ₐ comprises a light chain variable domain. In some embodiments, the light chain variable domain of H₁ₐ comprises a variable domain of an IgG1, IgG2, IgG3, or IgG4 light chain.

In some embodiments, the H₁ₐ binds to serum albumin selected from human serum albumin, cynomolgus serum albumin, and mouse serum albumin. In some embodiments, H₁ₐ binds to human serum albumin and cynomolgus serum albumin with comparable binding affinity (Kd). In some embodiments, H₁ₐ binds to mouse serum albumin with a binding affinity (Kd) that is about 1.5 fold to about 20 fold weaker than the binding affinity (Kd) of said protein towards human and cynomolgus serum albumin. In some embodiments, H₁ₐ binds to human serum albumin with a human Kd (hKd) between about 1 nM and about 100 nM and to cynomolgus serum albumin with a cynomolgus Kd (cKd) between 1 nM and 100 nM. In some embodiments, the hKd and the cKd of H₁ₐ are between 1 nM and about 5 nM, or about 5 nM and about 10 nM. In some embodiments, the hKd and cKd of H₁ₐ are between about 1 nM and about 2 nM, about 2 nM and about 3 nM, about 3 nM and about 4 nM, about 4 nM and about 5 nM, about 5 nM and about 6 nM, about 6 nM and about 7 nM, about 7 nM and about 8 nM, about 8 nM and about 9 nM, or about 9 nM and about 10 nM. In some embodiments, the ratio between the hKd and cKd (hKd:cKd) of H₁ₐ ranges from about 20:1 to about 1:2.

In some embodiments, H₁ₐ protein comprises elimination half-time of at least 12 hours, at least 20 hours, at least 25 hours, at least 30 hours, at least 35 hours, at least 40 hours, at least 45 hours, at least 50 hours, or at least 100 hours.

In some embodiments, H₁ or H₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments, H₁ or H₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cysteine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁ or H₁ₐ including the peptide backbone, the amino acid side chains, and the terminus.

TABLE 1
Half-life extending antibody or antibody fragment (H₁ or H₁ₐ)
(CDRs determined by IMGT definition)

| | Amino Acid Sequence | |
|---|---|---|
| Construct Description | (N to C) | SEQ ID NO: |
| Anti-Albumin: CDR-H1 | GSTFYTAV | 1 |
| Anti-Albumin: CDR-H2 | IRWTALTT | 2 |
| Anti-Albumin: CDR-H3 | AARGTLGLFTTADSYDY | 3 |
| (HE-1) Anti-albumin | EVQLVESGGGLVQPGGSLRLSCAASGSTF | 4 |
| | YTAVMGWVRQAPGKGLEWVAAIRWTA | |
| | LTTSYADSVKGRFTISRDGAKTTLYLQM | |
| | NSLRPEDTAVYYCAARGTLGLFTTADSY | |
| | DYWGQGTLVTVSS | |
| Anti-Albumin: CDR-H3 | AARGTLGLFTTADHYDY | 5 |
| (HE-2) Anti-albumin | EVQLVESGGGLVQPGGSLRLSCAASG | 6 |
| | STFYTAVMGWVRQAPGKGLEWVAA | |
| | IRWTALTTSYADSVKGRFTISRDGAK | |
| | TTLYLQMNSLRPEDTAVYYCAARGT | |
| | LGLFTTADHYDYWGQGTLVTVSS | |

Linking Moiety (L₃ or L₃ₐ)

In some embodiments, H₁ comprises a linking moiety (L₃) that connects H₁ to P₁. In some embodiments, H₁ₐ comprises a linking moiety (L₃ₐ) that connects H₁ₐ to P₁ₐ.

In some embodiments, L₃ or L₃ₐ is a peptide sequence having at least 5 to no more than 50 amino acids. In some embodiments, L₃ or L₃ₐ is a peptide sequence having at least 10 to no more than 30 amino acids. In some embodiments, L₃ or L₃ₐ is a peptide sequence having at least 10 amino acids. In some embodiments, L₃ or L₃ₐ is a peptide sequence having at least 18 amino acids. In some embodiments, L₃ or L₃ₐ is a peptide sequence having at least 26 amino acids.

In some embodiments, L₃ or L₃ₐ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1. In some embodiments, L₃ or L₃ₐ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of 1. In some embodiments, L₃ or L₃ₐ has a formula selected from the group consisting of (G₂S)ₙ (SEQ ID NO: 20), (GS)ₙ (SEQ ID NO: 36), (GSGGS)ₙ (SEQ ID NO: 37), (GGGS)ₙ (SEQ ID NO: 38), (GGGGS)ₙ (SEQ ID NO: 39), and (GSSGGS)ₙ (SEQ ID NO: 40), wherein n is an integer from 1 to 3.

In some embodiments, L₃ or L₃ₐ has a formula of (G₂S)ₙ, wherein n is an integer of least 1. In some embodiments, L₃ or L₃ₐ has a formula of (GS)ₙ, wherein n is an integer of least 1. In some embodiments, L₃ or L₃ₐ has a formula of (GSGGS)ₙ (SEQ ID NO: 15), wherein n is an integer of least 1. In some embodiments, L₃ or L₃ₐ has a formula of (GGGS)ₙ (SEQ ID NO: 16), wherein n is an integer of least 1. In some embodiments, L₃ or L₃ₐ has a formula of (GGGGS)ₙ (SEQ ID NO: 17), wherein n is an integer of least 1. In some embodiments, L₃ or L₃ₐ has a formula of (GSSGGS)ₙ (SEQ ID NO: 18), wherein n is an integer of least 1.

In some embodiments, L₃ or L₃ₐ has a formula of (G₂S)ₙ, wherein n is an integer of 1. In some embodiments, L₃ or L₃ₐ has a formula of (GS)ₙ, wherein n is an integer of 1. In some embodiments, L₃ or L₃ₐ has a formula of (GSGGS)ₙ (SEQ ID NO: 15), wherein n is an integer of 1. In some embodiments, L₃ or L₃ₐ has a formula of (GGGS)ₙ (SEQ ID NO: 16), wherein n is an integer of 1. In some embodiments, L₃ or L₃ₐ has a formula of (GGGGS)ₙ (SEQ ID NO: 17), wherein n is an integer of 1. In some embodiments, L₃ or L₃ₐ has a formula of (GSSGGS)ₙ (SEQ ID NO: 18), wherein n is an integer of 1.

In some embodiments, L₃ or L₃ₐ has a formula of (G₂S)ₙ (SEQ ID NO: 20), wherein n is an integer from 1 to 3. In some embodiments, L₃ or L₃ₐ has a formula of (GS)ₙ (SEQ ID NO: 36), wherein n is an integer from 1 to 3. In some embodiments, L₃ or L₃ₐ has a formula of (GSGGS)ₙ (SEQ ID NO: 37), wherein n is an integer from 1 to 3. In some embodiments, L₃ or L₃ₐ has a formula of (GGGS)ₙ (SEQ ID NO: 38), wherein n is an integer from 1 to 3. In some embodiments, L₃ or L₃ₐ has a formula of (GGGGS)ₙ (SEQ ID NO: 39), wherein n is an integer from 1 to 3. I In some embodiments, L₃ or L₃ₐ has a formula of (GSSGGS)ₙ (SEQ ID NO: 40), wherein n is an integer from 1 to 3.

### First Antigen Recognizing Molecule (A₁)

In some embodiments, A₁ comprises an antibody or antibody fragment. In some embodiments, A₁ comprises an antibody or antibody fragment that is human or humanized. In some embodiments, L₁ is bound to N-terminus of the antibody or antibody fragment of A₁. In some embodiments, A₂ is bound to N-terminus of the antibody or antibody fragment of A₁. In some embodiments, L₁ is bound to the C-terminus of the antibody or antibody fragment of A₁. In some embodiments, A₂ is bound to the C-terminus of the antibody or antibody fragment of A₁. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, the antibody or antibody fragment thereof comprises a single chain variable fragment (scFv), a heavy chain variable domain (VH domain), a light chain variable domain (VL domain), or a variable domain (VHH) of a camelid derived single domain antibody.

In some embodiments, the first target antigen comprises an effector cell antigen. In some embodiments, the first target antigen is cluster of differentiation 3 (CD3). In some embodiments, A₁ comprises an anti-CD3e single chain variable fragment. In some embodiments, A₁ comprises an anti-CD3e single chain variable fragment that has a KD binding of 1 μM or less to CD3 on CD3 expressing cells. In some embodiments, A₁ comprises a variable light chain and variable heavy chain each of which is capable of specifically binding to human CD3. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the polypeptide or polypeptide complex of formula I binds to an effector cell when L₁ is cleaved by the tumor specific protease. In some embodiments, the polypeptide or polypeptide complex of formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε.

In some embodiments, the first target antigen comprises a tumor antigen. In some embodiments, the first target antigen is epidermal growth factor receptor (EGFR), mesothelin, prostate-specific membrane antigen (PSMA), or tumor-associated calcium signal transducer 2 (TROP2). In some embodiments, A₁ the antibody or antibody fragment thereof of A₂ is humanized or human. In some embodiments, A₁ is a Fab or Fab′. In some embodiments, A₁ is wherein the Fab or Fab′ comprises (a) a Fab light chain polypeptide and (b) a Fab heavy chain polypeptide.

### Second Antigen Recognizing Molecule (A₂)

In some embodiments, A₂ comprises an antibody or antibody fragment. In some embodiments, the antibody or antibody fragment thereof of A₂ comprises a single chain variable fragment, a single domain antibody, Fab′, or a Fab. In some embodiments, the antibody or antibody fragment thereof of A₂ comprises a single chain variable fragment (scFv), a heavy chain variable domain (VH domain), a light chain variable domain (VL domain), a variable domain (VHH) of a camelid derived single domain antibody. In some embodiments, the antibody or antibody fragment thereof of A₂ is humanized or human. In some embodiments, is the Fab or Fab′. In some embodiments, the Fab or Fab′ comprises (a) a Fab light chain polypeptide and (b) a Fab heavy chain polypeptide. In some embodiments, the second target antigen comprises a tumor antigen. In some embodiments, the antibody or antibody fragment thereof comprises an epidermal growth factor receptor (EGFR) binding domain. In some embodiments, the antibody or antibody fragment thereof comprises a mesothelin binding domain. In some embodiments, the antibody or antibody fragment thereof comprises a PSMA binding domain. In some embodiments, the antibody or antibody fragment thereof comprises a TROP2 binding domain.

In some embodiments, the second target antigen comprises an effector cell antigen. In some embodiments, the second target antigen is CD3. In some embodiments, A₂ comprises an anti-CD3e single chain variable fragment. In some embodiments, A₂ comprises an anti-CD3e single chain variable fragment that has a KD binding of 1 μM or less to CD3 on CD3 expressing cells. In some embodiments, A₂ comprises a variable light chain and variable heavy chain each of which is capable of specifically binding to human CD3. In some embodiments, A₂ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the polypeptide or polypeptide complex of formula Ia binds to an effector cell when L₂ is cleaved by the tumor specific protease. In some embodiments, the polypeptide or polypeptide complex of formula I binds to an effector cell when L₂ is cleaved by the tumor specific protease and A₂ binds to the effector cell. In some embodiments, the effector cell is a T cell. In some embodiments, A₂ binds to a polypeptide that is part of a TCR-CD3 complex on the effector cell. In some embodiments, the polypeptide that is part of the TCR-CD3 complex is human CD3ε.

Peptide (P₁, P₁ₐ, or P₂)

In some embodiments, P₁ impairs binding of A₁ to the first target antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ has less than 70% sequence homology to the first target antigen. In some embodiments, P₁ has less than 70% sequence homology to the first target antigen. In some embodiments, P₁ has less than 75% sequence homology to the first target antigen. In some embodiments, P₁ has less than 80% sequence homology to the first target antigen. In some embodiments, P₁ has less than 85% sequence homology to the first target antigen. In some embodiments, P₁ has less than 90% sequence homology to the first target antigen. In some embodiments, P₁ has less than 95% sequence homology to the first target antigen. In some embodiments, P₁ has less than 98% sequence homology to the first target antigen. In some embodiments, P₁ has less than 99% sequence homology to the first target antigen.

In some embodiments, P₂ impairs binding of A₂ to the second target antigen. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ has less than 70% sequence homology to the second target antigen. In some embodiments, P₂ has less than 70% sequence homology to the second target antigen. In some embodiments, P₂ has less than 75% sequence homology to the second target antigen. In some embodiments, P₂ has less than 80% sequence homology to the second target antigen. In some embodiments, P₂ has less than 85% sequence homology to the second target antigen. In some embodiments, P₂ has less than 90% sequence homology to the second target antigen. In some embodiments, P₂ has less than 95% sequence homology to the second target antigen. In some embodiments, P₂ has less than 98% sequence homology to the second target antigen. In some embodiments, P₂ has less than 99% sequence homology to the second target antigen.

In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino acids in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide.

In some embodiments, P₁ₐ comprises a peptide sequence of at least 10 amino acids in length. In some embodiments, P₁ₐ comprises a peptide sequence of at least 10 amino acids in length and no more than 20 amino acids in length. In some embodiments, P₁ₐ comprises a peptide sequence of at least 16 amino acids in length. In some embodiments, P₁ₐ comprises a peptide sequence of no more than 40 amino acids in length. In some embodiments, P₁ₐ comprises at least two cysteine amino acid residues. In some embodiments, P₁ₐ comprises a cyclic peptide or a linear peptide. In some embodiments, P₁ₐ comprises a cyclic peptide. In some embodiments, P₁ₐ comprises a linear peptide. In some embodiments, P₁ₐ has less than 70% sequence homology to the effector cell antigen. In some embodiments, P₁ₐ has less than 75% sequence homology to the effector cell antigen. In some embodiments, P₁ₐ has less than 80% sequence homology to the effector cell antigen. In some embodiments, P₁ₐ has less than 85% sequence homology to the effector cell antigen. In some embodiments, P₁ₐ has less than 90% sequence homology to the effector cell antigen. In some embodiments, P₁ₐ has less than 95% sequence homology to the effector cell antigen. In some embodiments, P₁ₐ has less than 98% sequence homology to the effector cell antigen. In some embodiments, P₁ₐ has less than 99% sequence homology to the effector cell antigen.

In some embodiments, P₁, P₂, or P₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₂, or P₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁, P₂, or P₁ₐ including the peptide backbone, the amino acid side chains, and the terminus.

In some embodiments, P₁, P₂, or P₁ₐ does not comprise albumin or an albumin fragment. In some embodiments, P₁, P₂, or P₁ₐ does not comprise an albumin binding domain.

Cleavable Linker (L₁ or L₂)

In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids.

In some embodiments, L₁ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1. In some embodiments, L₁ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of 1. In some embodiments, L₁ has a formula selected from the group consisting of (G₂S)ₙ (SEQ ID NO: 20), (GS)ₙ (SEQ ID NO: 36), (GSGGS)ₙ (SEQ ID NO: 37), (GGGS)ₙ (SEQ ID NO: 38), (GGGGS)ₙ (SEQ ID NO: 39), and (GSSGGS)ₙ (SEQ ID NO: 40), wherein n is an integer from 1 to 3.

In some embodiments, L₁ has a formula of (G₂S), wherein n is an integer of least 1. In some embodiments, L₁ has a formula of (GS)ₙ, wherein n is an integer of least 1. In some embodiments, L₁ has a formula of (GSGGS)ₙ (SEQ ID NO: 15), wherein n is an integer of least 1. In some embodiments, L₁ has a formula of (GGGS)ₙ (SEQ ID NO: 16), wherein n is an integer of least 1. In some embodiments, L₁ has a formula of (GGGGS)ₙ (SEQ ID NO: 17), wherein n is an integer of least 1. In some embodiments, L₁ has a formula of (GSSGGS)ₙ (SEQ ID NO: 18), wherein n is an integer of least 1.

In some embodiments, L₁ has a formula of (G₂S), wherein n is an integer of 1. In some embodiments, L₁ has a formula of (GS)ₙ, wherein n is an integer of 1. In some embodiments, L₁ has a formula of (GSGGS)ₙ (SEQ ID NO: 15), wherein n is an integer of 1. In some embodiments, L₁ has a formula of (GGGS)ₙ (SEQ ID NO: 16), wherein n is an integer of 1. In some embodiments, L₁ has a formula of (GGGGS)ₙ (SEQ ID NO: 17), wherein n is an integer of 1. In some embodiments, L₁ has a formula of (GSSGGS)ₙ (SEQ ID NO: 18), wherein n is an integer of 1.

In some embodiments, L₁ has a formula of (G₂S)ₙ (SEQ ID NO: 20), wherein n is an integer from 1 to 3. In some embodiments, L₁ has a formula of (GS)ₙ (SEQ ID NO: 36), wherein n is an integer from 1 to 3. In some embodiments, L₁ has a formula of (GSGGS)ₙ (SEQ ID NO: 37), wherein n is an integer from 1 to 3. In some embodiments, L₁ has a formula of (GGGS)ₙ (SEQ ID NO: 38), wherein n is an integer from 1 to 3. In some embodiments, L₁ has a formula of (GGGGS)ₙ (SEQ ID NO: 39), wherein n is an integer from 1 to 3. In some embodiments, L₁ has a formula of (GSSGGS)ₙ (SEQ ID NO: 40), wherein n is an integer from 1 to 3.

In some embodiments, L₁ is a substrate for a tumor specific protease. In some embodiments, the tumor specific protease is selected from the group consisting of metalloprotease, serine protease, cysteine protease, threonine protease, and aspartic protease. In some embodiments, L₁ comprises a urokinase cleavable amino acid sequence, a matriptase cleavable amino acid sequence, a matrix metalloprotease cleavable amino acid sequence, or a legumain cleavable amino acid sequence. In some embodiments, L₁ comprises an amino acid sequence selected from the group consisting of GGGGSLSGRSDNHGSSGT (SEQ ID NO: 21), GGGGSSGGSGGSGLSGRSDNHGSSGT (SEQ ID NO: 22), ASGRSDNH (SEQ ID NO: 23), LAGRSDNH (SEQ ID NO: 24), ISSGLASGRSDNH (SEQ ID NO: 25), ISSGLLAGRSDNH (SEQ ID NO: 26), LSGRSDNH (SEQ ID NO: 27), ISSGLLSGRSDNP (SEQ ID NO: 28), ISSGLLSGRSDNH (SEQ ID NO: 29), LSGRSDNHSPLGLAGS (SEQ ID NO: 30), SPLGLAGSLSGRSDNH (SEQ ID NO: 31), SPLGLSGRSDNH (SEQ ID NO: 32), LAGRSDNHSPLGLAGS (SEQ ID NO: 33), LSGRSDNHVPLSLKMG (SEQ ID NO: 34), and LSGRSDNHVPLSLSMG (SEQ ID NO: 35). In some embodiments, L₁ comprises an amino acid sequence selected from the group consisting of ASGRSDNH (SEQ ID NO: 23), LAGRSDNH (SEQ ID NO: 24), ISSGLASGRSDNH (SEQ ID NO: 25), and ISSGLLAGRSDNH (SEQ ID NO: 26).

In some embodiments, L₂ has a formula selected from the group consisting of (G₂S)ₙ, (GS)ₙ, (GSGGS), (SEQ ID NO: 15) (GGGS)ₙ (SEQ ID NO: 16), (GGGGS)ₙ (SEQ ID NO: 17), and (GSSGGS)ₙ (SEQ ID NO: 18), wherein n is an integer of at least 1. In some embodiments, L₂ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of 1. In some embodiments, L₂ has a formula selected from the group consisting of (G₂S)ₙ (SEQ ID NO: 20), (GS)ₙ (SEQ ID NO: 36), (GSGGS)ₙ (SEQ ID NO: 37), (GGGS)ₙ (SEQ ID NO: 38), (GGGGS)ₙ (SEQ ID NO: 39), and (GSSGGS)ₙ (SEQ ID NO: 40), wherein n is an integer from 1 to 3.

In some embodiments, L₂ has a formula of (G₂S), wherein n is an integer of least 1. In some embodiments, L₂ has a formula of (GS)ₙ, wherein n is an integer of least 1. In some embodiments, L₂ has a formula of (GSGGS)ₙ (SEQ ID NO: 15), wherein n is an integer of least 1. In some embodiments, L₂ has a formula of (GGGS)ₙ (SEQ ID NO: 16), wherein n is an integer of least 1. In some embodiments, L₂ has a formula of (GGGGS)ₙ (SEQ ID NO: 17), wherein n is an integer of least 1. In some embodiments, L₂ has a formula of (GSSGGS)ₙ (SEQ ID NO: 18), wherein n is an integer of least 1.

In some embodiments, L₂ has a formula of (G₂S), wherein n is an integer of 1. In some embodiments, L₂ has a formula of (GS)ₙ, wherein n is an integer of 1. In some embodiments, L₂ has a formula of (GSGGS)ₙ (SEQ ID NO: 15), wherein n is an integer of 1. In some embodiments, L₂ has a formula of (GGGS)ₙ (SEQ ID NO: 16), wherein n is an integer of 1. In some embodiments, L₂ has a formula of (GGGGS)ₙ (SEQ ID NO: 17), wherein n is an integer of 1. In some embodiments, L₂ has a formula of (GSSGGS)ₙ (SEQ ID NO: 18), wherein n is an integer of 1.

In some embodiments, L₂ has a formula of (G₂S)ₙ (SEQ ID NO: 20), wherein n is an integer from 1 to 3. In some embodiments, L₂ has a formula of (GS)ₙ (SEQ ID NO: 36), wherein n is an integer from 1 to 3. In some embodiments, L₂ has a formula of (GSGGS)ₙ (SEQ ID NO: 37), wherein n is an integer from 1 to 3. In some embodiments, L₂ has a formula of (GGGS)ₙ (SEQ ID NO: 38), wherein n is an integer from 1 to 3. In some embodiments, L₂ has a formula of (GGGGS)ₙ (SEQ ID NO: 39), wherein n is an integer from 1 to 3. In some embodiments, L₂ has a formula of (GSSGGS)ₙ (SEQ ID NO: 40), wherein n is an integer from 1 to 3.

In some embodiments, L₂ is a substrate for a tumor specific protease. In some embodiments, the tumor specific protease is selected from the group consisting of metalloprotease, serine protease, cysteine protease, threonine protease, and aspartic protease. In some embodiments, L₂ comprises a urokinase cleavable amino acid sequence, a matriptase cleavable amino acid sequence, a matrix metalloprotease cleavable amino acid sequence, or a legumain cleavable amino acid sequence. In some embodiments, L₂ comprises an amino acid sequence selected from the group consisting of GGGGSLSGRSDNHGSSGT (SEQ ID NO: 21), GGGGSSGGSGGSGLSGRSDNHGSSGT (SEQ ID NO: 22), ASGRSDNH (SEQ ID NO: 23), LAGRSDNH (SEQ ID NO: 24), ISSGLASGRSDNH (SEQ ID NO: 25), ISSGLLAGRSDNH (SEQ ID NO: 26), LSGRSDNH (SEQ ID NO: 27), ISSGLLSGRSDNP (SEQ ID NO: 28), ISSGLLSGRSDNH (SEQ ID NO: 29), LSGRSDNHSPLGLAGS (SEQ ID NO: 30), SPLGLAGSLSGRSDNH (SEQ ID NO: 31), SPLGLSGRSDNH (SEQ ID NO: 32), LAGRSDNHSPLGLAGS (SEQ ID NO: 33), LSGRSDNHVPLSLKMG (SEQ ID NO: 34), and LSGRSDNHVPLSLSMG (SEQ ID NO: 35). In some embodiments, L₂ comprises an amino acid sequence selected from the group consisting of ASGRSDNH (SEQ ID NO:23), LAGRSDNH (SEQ ID NO: 24), ISSGLASGRSDNH (SEQ ID NO: 25), and ISSGLLAGRSDNH (SEQ ID NO: 26).

In some embodiments, L₁ or L₂ comprises a modified amino acid or non-natural amino acid, or a modified non-natural amino acid, or a combination thereof. In some embodiments, the modified amino acid or a modified non-natural amino acid comprises a post-translational modification. In some embodiments, L₁ or L₂ comprises a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cysteine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In some embodiments, modifications are made anywhere to L₁ or L₂ including the peptide backbone, or the amino acid side chains.

### Configurations

In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab light chain polypeptide of A₂ is bound to the scFv light chain polypeptide of A₁. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

Disclosed herein, in some embodiments, are polypeptides or polypeptide complexes comprising a structural arrangement according to Configuration 1:

wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety (L₁) that is a substrate for a tumor specific protease, and P₁ is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3; and a Fab that binds to a tumor cell antigen, wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease.

Disclosed herein, in some embodiments, are polypeptides or polypeptide complexes comprising a structural arrangement according to Configuration 2:

wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Disclosed herein, in some embodiments, are polypeptides or polypeptide complexes comprising a structural arrangement according to Configuration 3:

wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are polypeptides or polypeptide complexes comprising a structural arrangement according to Configuration 4:

wherein the polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv.

Disclosed herein, in some embodiments, are polypeptides or polypeptide complexes comprising a structural arrangement according to Configuration 5:

wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab light chain polypeptide to P₂ and is a substrate for a tumor specific protease.

Disclosed herein, in some embodiments, are polypeptides or polypeptide complexes comprising a structural arrangement according to Configuration 6:

wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the heavy chain variable domain of the scFv with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are polypeptides or polypeptide complexes comprising a structural arrangement according to Configuration 7:

wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are polypeptides or polypeptide complexes comprising a structural arrangement according to Configuration 8:

wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv.

Disclosed herein, in some embodiments, are polypeptides or polypeptide complexes comprising a structural arrangement according to Configuration 9:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and the P₁ is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to Configuration 10:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide.

Disclosed herein, in some embodiments, are polypeptides or polypeptide complexes comprising a structural arrangement according to Configuration 11:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab heavy chain polypeptide with a linking moiety (L₁) that is a substrate for a tumor specific protease, and P₁
is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to Configuration 12:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide.

Disclosed herein, in some embodiments, are polypeptides or polypeptide complexes comprising a structural arrangement according to Configuration 13:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and P₁
is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to Configuration 14:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide.

Disclosed herein, in some embodiments, are polypeptides or polypeptide complexes comprising a structural arrangement according to Configuration 15:

wherein the polypeptide or polypeptide complex comprises a Fab that binds to a tumor cell antigen, the Fab comprising a Fab light chain polypeptide and a Fab heavy chain polypeptide, wherein the Fab is linked to a (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to Configuration 16:

wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending single domain antibody (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide.

### Polynucleotides Encoding Polypeptides or Polypeptide Complexes

Disclosed herein, in some embodiments, are isolated recombinant nucleic acid molecules encoding polypeptides or polypeptide complexes as disclosed herein. In some embodiments, the polypeptides or polypeptide complexes comprise an antibody or an antibody fragment.

Disclosed herein, in some embodiments, are isolated recombinant nucleic acid molecules encoding a polypeptide or polypeptide complex comprising a half-life extending antibody or antibody fragment (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3.

In some embodiments, is an isolated nucleic acid encoding an amino acid sequence with at least 90% sequence identity to SEQ ID NO: 4. In some embodiments, is an isolated nucleic acid encoding an amino acid sequence with at least 95% sequence identity to SEQ ID NO: 4. In some embodiments, is an isolated nucleic acid encoding an amino acid sequence with at least 99% sequence identity to SEQ ID NO: 4. In some embodiments, is an isolated nucleic acid encoding an amino acid sequence according to SEQ ID NO: 4.

In some embodiments, is an isolated nucleic acid encoding an amino acid sequence of at least 110 consecutive amino acid residues of SEQ ID NO: 4. In some embodiments, is an isolated nucleic acid encoding an amino acid sequence of at least 120 consecutive amino acid residues of SEQ ID NO: 4. In some embodiments, is an isolated nucleic acid encoding an amino acid sequence of at least 110 consecutive amino acid residues of SEQ ID NO: 4 and has at least 95% sequence identity to the at least 110 consecutive amino acid residues of SEQ ID NO: 4. In some embodiments, is an isolated nucleic acid encoding an amino acid sequence of at least 120 consecutive amino acid residues of SEQ ID NO: 4 and has at least 95% sequence identity to the at least 120 consecutive amino acid residues of SEQ ID NO: 4.

Disclosed herein, in some embodiments, are isolated recombinant nucleic acid molecules encoding polypeptide or polypeptide complex comprising a half-life extending antibody or antibody fragment (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 5; and wherein the CDRs comprise from 0-2 amino acid modifications in at least one of the HC-CDR1, HC-CDR2, or HC-CDR3.

In some embodiments, is an isolated nucleic acid encoding an amino acid sequence with at least 90% sequence identity to SEQ ID NO: 6. In some embodiments, is an isolated nucleic acid encoding an amino acid sequence with at least 95% sequence identity to SEQ ID NO: 6. In some embodiments, is an isolated nucleic acid encoding an amino acid sequence with at least 99% sequence identity to SEQ ID NO: 6. In some embodiments, is an isolated nucleic acid encoding an amino acid sequence according to SEQ ID NO: 6.

In some embodiments, is an isolated nucleic acid encoding an amino acid sequence of at least 110 consecutive amino acid residues of SEQ ID NO: 6. In some embodiments, is an isolated nucleic acid encoding an amino acid sequence of at least 120 consecutive amino acid residues of SEQ ID NO: 6. In some embodiments, is an isolated nucleic acid encoding an amino acid sequence of at least 110 consecutive amino acid residues of SEQ ID NO: 6 and has at least 95% sequence identity to the at least 110 consecutive amino acid residues of SEQ ID NO: 6. In some embodiments, is an isolated nucleic acid encoding an amino acid sequence of at least 120 consecutive amino acid residues of SEQ ID NO: 6 and has at least 95% sequence identity to the at least 120 consecutive amino acid residues of SEQ ID NO: 6.

### Pharmaceutical Compositions

Disclosed herein, in some embodiments, are pharmaceutical compositions comprising: (a) the polypeptides or polypeptide complexes as disclosed herein; and (b) a pharmaceutically acceptable excipient.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3 xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3 xFLAG-CMV 7.1, pFLAG-CMV 20, p3 xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3 xFLAG-CMV 9, p3 xFLAG-CMV 13, pFLAG-Myc-CMV 21, p3 xFLAG-Myc-CMV 25, pFLAG-CMV 4, p3 xFLAG-CMV 10, p3 xFLAG-CMV 14, pFLAG-Myc-CMV 22, p3 xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), an animal cell or a plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™ TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to a tumor antigen defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Embodiments

Embodiment 1 comprises an isolated polypeptide or polypeptide complex comprising a half-life extending antibody or antibody fragment (H₁) that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3.

Embodiment 2 comprises an isolated polypeptide or polypeptide complex of embodiment 1, wherein H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3.

Embodiment 3 comprises an isolated polypeptide or polypeptide complex of embodiment 1 or 2, wherein the antibody or antibody fragment of H₁ comprises a single domain antibody, a single chain variable fragment, a Fab, or Fab′.

Embodiment 4 comprises an isolated polypeptide or polypeptide complex of embodiment 3, wherein the antibody or antibody fragment of H₁ comprises the single domain antibody.

Embodiment 5 comprises an isolated polypeptide or polypeptide complex of any one of embodiments 1-4, wherein H₁ comprises an amino acid sequence with at least 90% sequence identity to SEQ ID NO: 4.

Embodiment 6 comprises an isolated polypeptide or polypeptide complex of any one of embodiments 1-5, wherein H₁ comprises an amino acid sequence with at least 95% sequence identity to SEQ ID NO: 4.

Embodiment 7 comprises an isolated polypeptide or polypeptide complex of any one of embodiments 1-6, wherein H₁ comprises an amino acid sequence with at least 99% sequence identity to SEQ ID NO: 4.

Embodiment 8 comprises an isolated polypeptide or polypeptide complex of any one of embodiments 1-7, wherein H₁ comprises an amino acid sequence according to SEQ ID NO: 4.

Embodiment 9 comprises an isolated polypeptide or polypeptide complex of any one of embodiments 1-4, wherein H₁ comprises an amino acid sequence of at least 110 consecutive amino acid residues of SEQ ID NO: 4.

Embodiment 10 comprises an isolated polypeptide or polypeptide complex of any one of embodiments 1-4, wherein H₁ comprises an amino acid sequence of at least 120 consecutive amino acid residues of SEQ ID NO: 4.

Embodiment 11 comprises an isolated polypeptide or polypeptide complex of any one of embodiments 1-4, wherein H₁ comprises an amino acid sequence of at least 110 consecutive amino acid residues of SEQ ID NO: 4 and has at least 95% sequence identity to the at least 110 consecutive amino acid residues of SEQ ID NO: 4.

Embodiment 12 comprises an isolated polypeptide or polypeptide complex of any one of embodiments 1-4, wherein H₁ comprises an amino acid sequence of at least 120 consecutive amino acid residues of SEQ ID NO: 4 and has at least 95% sequence identity to the at least 120 consecutive amino acid residues of SEQ ID NO: 4.

Embodiment 13 comprises an isolated polypeptide or polypeptide complex of any one of embodiments 1-12, wherein H₁ is linked to a peptide (P₁) that impairs binding of a first antigen recognizing molecule (A₁) to a first target antigen through a cleavable linker (L₁) that is a substrate for a tumor specific protease in a configuration according to Formula I: A₁-L₁-P₁-H₁

Embodiment 14 comprises an isolated polypeptide or polypeptide complex of embodiment 13, wherein A₁ is further linked to a second antigen recognizing molecule (A₂).

Embodiment 15 comprises an isolated polypeptide or polypeptide complex of embodiment 14, wherein the polypeptide or polypeptide complex is according to Formula Ia: P₂-L₂-A₂-A₁-L₁-P₁-H₁ wherein P₂ comprises a peptide that impairs binding of A₂ to a second target antigen; and L₂ comprises a second cleavable linker that connects A₂ to P₂ and is a substrate for a tumor specific protease.

Embodiment 16 comprises an isolated polypeptide of any one of embodiments 1-15, wherein H₁ comprises a linking moiety (L₃) that connects H₁ to P₁.

Embodiment 17 comprises an isolated polypeptide of embodiment 16, wherein L₃ is a peptide sequence having at least 5 to no more than 50 amino acids.

Embodiment 18 comprises an isolated polypeptide of embodiment 16, wherein L₃ is a peptide sequence having at least 10 to no more than 30 amino acids.

Embodiment 19 comprises an isolated polypeptide of embodiment 16, wherein L₃ is a peptide sequence having at least 10 amino acids.

Embodiment 20 comprises an isolated polypeptide of embodiment 16, wherein L₃ is a peptide sequence having at least 18 amino acids.

Embodiment 21 comprises an isolated polypeptide of embodiment 16, wherein L₃ is a peptide sequence having at least 26 amino acids.

Embodiment 22 comprises an isolated polypeptide of embodiment 16, wherein L₃ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1.

Embodiment 23 comprises an isolated polypeptide of embodiment 22, wherein L₃ comprises an amino acid sequence GGGGSGGGS (SEQ ID NO: 19).

Embodiment 24 comprises an isolated polypeptide or polypeptide complex of any one of embodiments 13-23, wherein A₁ comprises an antibody or antibody fragment.

Embodiment 25 comprises an isolated polypeptide or polypeptide complex of embodiment 24, wherein A₁ comprises an antibody or antibody fragment that is human or humanized.

Embodiment 26 comprises an isolated polypeptide or polypeptide complex of any one of embodiments 13-25, wherein L₁ is bound to N-terminus of the antibody or antibody fragment of A₁.

Embodiment 27 comprises an isolated polypeptide or polypeptide complex of any one of embodiments 15-25, wherein A₂ is bound to N-terminus of the antibody or antibody fragment of A₁.

Embodiment 28 comprises an isolated polypeptide or polypeptide complex of any one of embodiments 13-25, wherein L₁ is bound to the C-terminus of the antibody or antibody fragment of A₁.

Embodiment 29 comprises an isolated polypeptide or polypeptide complex of any one of embodiments 15-25, wherein A₂ is bound to the C-terminus of the antibody or antibody fragment of A₁.

Embodiment 30 comprises an isolated polypeptide or polypeptide complex of any one of embodiments 13-29, wherein the antibody or antibody fragment of A₁ comprises a single chain variable fragment, a single domain antibody, or a Fab fragment.

Embodiment 31 comprises an isolated polypeptide or polypeptide complex of embodiment 30, wherein A₁ is the single chain variable fragment (scFv).

Embodiment 32 comprises an isolated polypeptide or polypeptide complex of embodiment 31, wherein the scFv comprises a scFv heavy chain polypeptide and a scFv light chain polypeptide.

Embodiment 33 comprises an isolated polypeptide or polypeptide complex of embodiment 30, wherein A₁ is the single domain antibody.

Embodiment 34 comprises an isolated polypeptide or polypeptide complex of embodiment 30, wherein the antibody or antibody fragment thereof of A₁ comprises a single chain variable fragment (scFv), a heavy chain variable domain (VH domain), a light chain variable domain (VL domain), or a variable domain (VHH) of a camelid derived single domain antibody.

Embodiment 35 comprises an isolated polypeptide or polypeptide complex of any one of embodiments 13-34, wherein the first target antigen comprises an effector cell antigen.

Embodiment 36 comprises an isolated polypeptide or polypeptide complex of any one of embodiments 13-35, wherein the first target antigen is CD3.

Embodiment 37 comprises an isolated polypeptide or polypeptide complex of any one of embodiments 13-36, wherein A₁ comprises an anti-CD3e single chain variable fragment.

Embodiment 38 comprises an isolated polypeptide or polypeptide complex of any one of embodiments 13-37, wherein A₁ comprises an anti-CD3e single chain variable fragment that has a KD binding of 1 μM or less to CD3 on CD3 expressing cells.

Embodiment 39 comprises an isolated polypeptide or polypeptide complex of any one of embodiments 13-38, wherein A₁ comprises a variable light chain and variable heavy chain each of which is capable of specifically binding to human CD3.

Embodiment 40 comprises an isolated polypeptide or polypeptide complex of any one of embodiments 13-39, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 41 comprises an isolated polypeptide or polypeptide complex of any one of embodiments 13-40, wherein the polypeptide or polypeptide complex of formula I binds to an effector cell when L₁ is cleaved by the tumor specific protease.

Embodiment 42 comprises an isolated polypeptide or polypeptide complex of any one of embodiments 13-40, wherein the polypeptide or polypeptide complex of formula I binds to an effector cell when L₁ is cleaved by the tumor specific protease and A₁ binds to the effector cell.

Embodiment 43 comprises an isolated polypeptide or polypeptide complex of embodiment 42, wherein the effector cell is a T cell.

Embodiment 44 comprises an isolated polypeptide or polypeptide complex of embodiment 43, wherein A₁ binds to a polypeptide that is part of a TCR-CD3 complex on the effector cell.

Embodiment 45 comprises an isolated polypeptide or polypeptide complex of embodiment 44, wherein the polypeptide that is part of the TCR-CD3 complex is human CD3ε.

Embodiment 46 comprises an isolated polypeptide or polypeptide complex of any one of embodiments 15-45, wherein A₂ comprises an antibody or antibody fragment.

Embodiment 47 comprises an isolated polypeptide or polypeptide complex of embodiment 46, wherein the antibody or antibody fragment of A₂ comprises a single chain variable fragment, a single domain antibody, Fab′, or a Fab.

Embodiment 48 comprises an isolated polypeptide or polypeptide complex of embodiment 46, wherein the antibody or antibody fragment of A₂ comprises a single chain variable fragment (scFv), a heavy chain variable domain (VH domain), a light chain variable domain (VL domain), or a variable domain (VHH) of a camelid derived single domain antibody.

Embodiment 49 comprises an isolated polypeptide or polypeptide complex of any one of embodiments 46-48, wherein the antibody or antibody fragment thereof of A₂ is humanized or human.

Embodiment 50 comprises an isolated polypeptide or polypeptide complex of embodiment 47, wherein A₂ is the Fab or Fab′.

Embodiment 51 comprises an isolated polypeptide or polypeptide complex of embodiment 50, wherein the Fab or Fab′ comprises (a) a Fab light chain polypeptide and (b) a Fab heavy chain polypeptide.

Embodiment 52 comprises an isolated polypeptide or polypeptide complex of any one of embodiments 15-51, wherein the second target antigen comprises a tumor antigen.

Embodiment 53 comprises an isolated polypeptide or polypeptide complex of any one of embodiments 46-52, wherein the antibody or antibody fragment of A₂ thereof comprises an epidermal growth factor receptor (EGFR) binding domain, a mesothelin binding domain, PSMA binding domain, or a TROP2 binding domain.

Embodiment 54 comprises an isolated polypeptide or polypeptide complex of any one of embodiments 46-52, wherein the antibody or antibody fragment comprises a PSMA binding domain or a TROP2 binding domain.

Embodiment 55 comprises an isolated polypeptide or polypeptide complex of any one of embodiments 13-54, wherein P₁ impairs binding of A₁ to the first target antigen.

Embodiment 56 comprises an isolated polypeptide or polypeptide complex of any one of embodiments 13-55, wherein P₁ is bound to A₁ through ionic interactions, electrostatic interactions, hydrophobic interactions, Pi-stacking interactions, and or H-bonding interactions, or a combination thereof.

Embodiment 57 comprises an isolated polypeptide or polypeptide complex of any one of embodiments 13-56, wherein P₁ has less than 70% sequence homology to the first target antigen.

Embodiment 58 comprises an isolated polypeptide or polypeptide complex of any one of embodiments 15-57, wherein P₂ impairs binding of A₂ to the second target antigen.

Embodiment 59 comprises an isolated polypeptide or polypeptide complex of any one of embodiments 15-58, wherein P₂ is bound to A₂ through ionic interactions, electrostatic interactions, hydrophobic interactions, Pi-stacking interactions, or H-bonding interactions, or a combination thereof.

Embodiment 60 comprises an isolated polypeptide or polypeptide complex of any one of embodiments 15-59, wherein P₂ is bound to A₂ at or near an antigen binding site.

Embodiment 61 comprises an isolated polypeptide or polypeptide complex of any one of embodiments 15-60, wherein P₂ has less than 70% sequence homology to the second target antigen.

Embodiment 62 comprises an isolated polypeptide or polypeptide complex of any one of embodiments 13-61, wherein P₁ or P₂ comprises a peptide sequence of at least 10 amino acids in length.

Embodiment 63 comprises an isolated polypeptide or polypeptide complex of any one of embodiments 13-61, wherein P₁ or P₂ comprises a peptide sequence of at least 10 amino acids in length and no more than 20 amino acids in length.

Embodiment 64 comprises an isolated polypeptide or polypeptide complex of any one of embodiments 13-61, wherein P₁ or P₂ comprises a peptide sequence of at least 16 amino acids in length.

Embodiment 65 comprises an isolated polypeptide or polypeptide complex of any one of embodiments 13-61, wherein P₁ or P₂ comprises a peptide sequence of no more than 40 amino acids in length.

Embodiment 66 comprises an isolated polypeptide or polypeptide complex of any one of embodiments 13-61, wherein P₁ or P₂ comprises at least two cysteine amino acid residues.

Embodiment 67 comprises an isolated polypeptide or polypeptide complex of any one of embodiments 13-61, wherein P₁ or P₂ comprises a cyclic peptide or a linear peptide.

Embodiment 68 comprises an isolated polypeptide or polypeptide complex of any one of embodiments 13-61, wherein P₁ or P₂ comprises a cyclic peptide.

Embodiment 69 comprises an isolated polypeptide or polypeptide complex of any one of embodiments 13-61, wherein P₁ or P₂ comprises a linear peptide

Embodiment 70 comprises an isolated polypeptide or polypeptide complex of any one of embodiments 13-69, wherein L₁ is bound to N-terminus of A₁.

Embodiment 71 comprises an isolated polypeptide or polypeptide complex of any one of embodiments 13-69, wherein L₁ is bound to C-terminus of A₁.

Embodiment 72 comprises an isolated polypeptide or polypeptide complex of any one of embodiments 15-69, wherein L₂ is bound to N-terminus of A₂.

Embodiment 73 comprises an isolated polypeptide or polypeptide complex of any one of embodiments 15-69, wherein L₂ is bound to C-terminus of A₂.

Embodiment 74 comprises an isolated polypeptide or polypeptide complex of any one of embodiments 13-73, wherein L₁ or L₂ is a peptide sequence having at least 5 to no more than 50 amino acids.

Embodiment 75 comprises an isolated polypeptide or polypeptide complex of any one of embodiments 13-73, wherein L₁ or L₂ is a peptide sequence having at least 10 to no more than 30 amino acids.

Embodiment 76 comprises an isolated polypeptide or polypeptide complex of any one of embodiments 13-73, wherein L₁ or L₂ is a peptide sequence having at least 10 amino acids.

Embodiment 77 comprises an isolated polypeptide or polypeptide complex of any one of embodiments 13-73, wherein L₁ or L₂ is a peptide sequence having at least 18 amino acids.

Embodiment 78 comprises an isolated polypeptide or polypeptide complex of any one of embodiments 13-73, wherein L₁ or L₂ is a peptide sequence having at least 26 amino acids.

Embodiment 79 comprises an isolated polypeptide or polypeptide complex of any one of embodiments 13-73, wherein L₁ or L₂ has a formula comprising (G₂S)ₙ (SEQ ID NO: 20), wherein n is an integer from 1 to 3.

Embodiment 80 comprises an isolated polypeptide or polypeptide complex of any one of embodiments 13-73, wherein L₁ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1.

Embodiment 81 comprises an isolated polypeptide or polypeptide complex of any one of embodiments 13-80, wherein P₁ becomes unbound from A₁ when L₁ is cleaved by the tumor specific protease thereby exposing A₁ to the first target antigen.

Embodiment 82 comprises an isolated polypeptide or polypeptide complex of any one of embodiments 15-81, wherein P₂ becomes unbound from A₂ when L₂ is cleaved by the tumor specific protease thereby exposing A₂ to the second target antigen.

Embodiment 83 comprises an isolated polypeptide or polypeptide complex of any one of embodiments 13-82, wherein the tumor specific protease is selected from the group consisting of metalloprotease, serine protease, cysteine protease, threonine protease, and aspartic protease.

Embodiment 84 comprises an isolated polypeptide or polypeptide complex of any one of embodiments 13-82, wherein L₁ or L₂ comprises a urokinase cleavable amino acid sequence, a matriptase cleavable amino acid sequence, matrix metalloprotease cleavable amino acid sequence, or a legumain cleavable amino acid sequence.

Embodiment 85 comprises an isolated polypeptide or polypeptide complex of any one of embodiments 13-84, wherein L₁ or L₂ comprises an amino acid sequence selected from the group consisting of GGGGSLSGRSDNHGSSGT (SEQ ID NO: 21), GGGGSSGGSGGSGLSGRSDNHGSSGT (SEQ ID NO: 22), ASGRSDNH (SEQ ID NO: 23), LAGRSDNH (SEQ ID NO: 24), ISSGLASGRSDNH (SEQ ID NO: 25), ISSGLLAGRSDNH (SEQ ID NO: 26), LSGRSDNH (SEQ ID NO: 27), ISSGLLSGRSDNP (SEQ ID NO: 28), ISSGLLSGRSDNH (SEQ ID NO: 29), LSGRSDNHSPLGLAGS (SEQ ID NO: 30), SPLGLAGSLSGRSDNH (SEQ ID NO: 31), and SPLGLSGRSDNH (SEQ ID NO: 32).

Embodiment 86 comprises an isolated polypeptide or polypeptide complex of any one of embodiments 13-84, wherein L₁ or L₂ comprises an amino acid sequence selected from the group consisting of ASGRSDNH (SEQ ID NO: 23), LAGRSDNH (SEQ ID NO: 24), ISSGLASGRSDNH (SEQ ID NO: 25), and ISSGLLAGRSDNH (SEQ ID NO: 26).

Embodiment 87 comprises an isolated polypeptide or polypeptide complex of any one of embodiments 47-86, wherein the Fab light chain polypeptide of A₂ is bound to a C-terminus of the single chain variable fragment (scFv) of A₁.

Embodiment 88 comprises an isolated polypeptide or polypeptide complex of any one of embodiments 47-86, wherein the Fab heavy chain polypeptide of A₂ is bound to a C-terminus of the single chain variable fragment (scFv) A₁.

Embodiment 89 comprises an isolated polypeptide or polypeptide complex of any one of embodiments 47-86, wherein the Fab light chain polypeptide of A₂ is bound to a N-terminus of the single chain variable fragment (scFv) of A₁.

Embodiment 90 comprises an isolated polypeptide or polypeptide complex of any one of embodiments 47-86, wherein the Fab heavy chain polypeptide of A₂ is bound to a N-terminus of the single chain variable fragment (scFv) of A₁.

Embodiment 91 comprises an isolated polypeptide or polypeptide complex of any one of embodiments 47-86, wherein the Fab heavy chain polypeptide of A₂ is bound to the scFv heavy chain polypeptide of A₁.

Embodiment 92 comprises an isolated polypeptide or polypeptide complex of any one of embodiments 47-86, wherein the Fab light chain polypeptide of A₂ is bound to the scFv heavy chain polypeptide of A₁.

Embodiment 93 comprises an isolated polypeptide or polypeptide complex of any one of embodiments 47-86, wherein the Fab heavy chain polypeptide of A₂ is bound to the scFv light chain polypeptide of A₁.

Embodiment 94 comprises an isolated polypeptide or polypeptide complex of any one of embodiments 47-86, wherein the Fab light chain polypeptide of A₂ is bound to the scFv light chain polypeptide of A₁.

Embodiment 95 comprises an isolated polypeptide or polypeptide complex of any one of embodiments 47-86, wherein the Fab heavy chain polypeptide of A₂ is bound to the scFv heavy chain polypeptide of A₁ and L₂ is bound to the Fab light chain polypeptide of A₂.

Embodiment 96 comprises an isolated polypeptide or polypeptide complex of any one of embodiments 47-86, wherein the Fab light chain polypeptide of A₂ is bound to the scFv heavy chain polypeptide of A₁ and L₂ is bound to the Fab heavy chain polypeptide of A₂.

Embodiment 97 comprises an isolated polypeptide or polypeptide complex of any one of embodiments 47-86, wherein the Fab heavy chain polypeptide of A₂ is bound to the scFv light chain polypeptide of A₁ and L₂ is bound to the Fab light chain polypeptide of A₂.

Embodiment 98 comprises an isolated polypeptide or polypeptide complex of any one of embodiments 47-86, wherein the Fab light chain polypeptide of A₂ is bound to the scFv light chain polypeptide of A₁ and L₂ is bound to the Fab heavy chain polypeptide of A₂.

Embodiment 99 comprises a pharmaceutical composition comprising: (a) the polypeptide or polypeptide complex of any one of embodiments 1-98; and (b) a pharmaceutically acceptable excipient.

Embodiment 100 comprises an isolated recombinant nucleic acid molecule encoding the polypeptide or polypeptide complex of any one of embodiments 1-98.

Embodiment 101 comprises an isolated polypeptide or polypeptide complex according to Formula II: L₁ₐ-P₁ₐ-H₁ₐ wherein L₁ₐ comprises a tumor specific protease-cleaved linking moiety that when uncleaved connects P₁ₐ to a first antigen recognizing molecule that binds to a first target antigen; P₁ₐ comprises a peptide that impairs binding of the first antigen recognizing molecule to the first target antigen when L₁ₐ is uncleaved; and H₁ₐ comprises a half-life extending molecule that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3.

Embodiment 102 comprises an isolated polypeptide or polypeptide complex of embodiment 101, wherein H₁ₐ comprises complementarity determining regions (CDRs): HC-CDR1, HC-CDR2, and HC-CDR3, wherein the HC-CDR1, the HC-CDR2, and the HC-CDR3 of H₁ₐ comprise: HC-CDR1: SEQ ID NO: 1, HC-CDR2: SEQ ID NO: 2, and HC-CDR3: SEQ ID NO: 3.

Embodiment 103 comprises an isolated polypeptide or polypeptide complex of embodiment 101 or 102, wherein the antibody or antibody fragment of H₁ₐ comprises a single domain antibody, a single chain variable fragment, a Fab, or Fab′.

Embodiment 104 comprises an isolated polypeptide or polypeptide complex of embodiment 103, wherein the antibody or antibody fragment of H₁ comprises the single domain antibody.

Embodiment 105 comprises an isolated polypeptide or polypeptide complex of any one of embodiment 101-104, wherein H₁ₐ comprises an amino acid sequence with at least 90% sequence identity to SEQ ID NO: 4.

Embodiment 106 comprises an isolated polypeptide or polypeptide complex of any one of embodiments 101-105, wherein H₁ₐ comprises an amino acid sequence with at least 95% sequence identity to SEQ ID NO: 4.

Embodiment 107 comprises an isolated polypeptide or polypeptide complex of any one of embodiments 101-106, wherein H₁ₐ comprises an amino acid sequence with at least 99% sequence identity to SEQ ID NO: 4.

Embodiment 108 comprises an isolated polypeptide or polypeptide complex of any one of embodiments 101-107, wherein H₁ₐ comprises an amino acid sequence according to SEQ ID NO: 4.

Embodiment 109 comprises an isolated polypeptide or polypeptide complex of any one of embodiments 101-104, wherein H₁ₐ comprises an amino acid sequence of at least 110 consecutive amino acid residues of SEQ ID NO: 4.

Embodiment 110 comprises an isolated polypeptide or polypeptide complex of any one of embodiments 101-104, wherein H₁ₐ comprises an amino acid sequence of at least 120 consecutive amino acid residues of SEQ ID NO: 4.

Embodiment 111 comprises an isolated polypeptide or polypeptide complex of any one of embodiments 101-104, wherein H₁ₐ comprises an amino acid sequence of at least 110 consecutive amino acid residues of SEQ ID NO: 4 and has at least 95% sequence identity to the at least 110 consecutive amino acid residues of SEQ ID NO: 4.

Embodiment 112 comprises an isolated polypeptide or polypeptide complex of any one of embodiments 101-104, wherein H₁ₐ comprises an amino acid sequence of at least 120 consecutive amino acid residues of SEQ ID NO: 4 and has at least 95% sequence identity to the at least 120 consecutive amino acid residues of SEQ ID NO: 4.

Embodiment 113 comprises an isolated polypeptide or polypeptide complex of any one of embodiments 101-104, wherein P₁ₐ when L₁ₐ is uncleaved impairs binding of the first antigen recognizing molecule to the effector cell antigen antigen.

Embodiment 114 comprises an isolated polypeptide or polypeptide complex of any one of embodiments 101-104, wherein the first antigen recognizing molecule comprises an antibody or antibody fragment.

Embodiment 115 comprises an isolated polypeptide or polypeptide complex of embodiment 113, wherein the effector cell antigen is an anti-CD3 effector cell antigen.

Embodiment 116 comprises an isolated polypeptide or polypeptide complex of embodiment 113 or 115, wherein P₁ₐ has less than 70% sequence homology to the effector cell antigen.

Embodiment 117 comprises an isolated polypeptide or polypeptide complex of any one of embodiments 101-116, wherein P₁ₐ comprises a peptide sequence of at least 10 amino acids in length.

Embodiment 118 comprises an isolated polypeptide or polypeptide complex of any one of embodiments 101-116, wherein P₁ₐ comprises a peptide sequence of at least 10 amino acids in length and no more than 20 amino acids in length.

Embodiment 119 comprises an isolated polypeptide or polypeptide complex of any one of embodiments 101-116, wherein P₁ₐ comprises a peptide sequence of at least 16 amino acids in length.

Embodiment 120 comprises an isolated polypeptide or polypeptide complex of any one of embodiments 101-116, wherein P₁ₐ comprises a peptide sequence of no more than 40 amino acids in length.

Embodiment 121 comprises an isolated polypeptide or polypeptide complex of any one of embodiments 101-120, wherein P₁ₐ comprises at least two cysteine amino acid residues.

Embodiment 122 comprises an isolated polypeptide or polypeptide complex of any one of embodiments 101-121, wherein P₁ₐ comprises a cyclic peptide or a linear peptide.

Embodiment 123 comprises an isolated polypeptide or polypeptide complex of any one of embodiments 101-122, wherein P₁ₐ comprises a cyclic peptide.

Embodiment 124 comprises an isolated polypeptide or polypeptide complex of any one of embodiments 101-122, wherein P₁ₐ comprises a linear peptide.

Embodiment 125 comprises an isolated polypeptide or polypeptide complex of any one of embodiments 101-124, wherein H₁ₐ comprises a linking moiety (L₃ₐ) that connects H₁ₐ to P₁ₐ.

Embodiment 126 comprises an isolated polypeptide or polypeptide complex of any one of embodiments 101-125, wherein L₃ₐ is a peptide sequence having at least 5 to no more than 50 amino acids.

Embodiment 127 comprises an isolated polypeptide or polypeptide complex of any one of embodiments 101-126, wherein L₃ₐ is a peptide sequence having at least 10 to no more than 30 amino acids.

Embodiment 128 comprises an isolated polypeptide or polypeptide complex of any one of embodiments 101-125, wherein L₃ₐ is a peptide sequence having at least 10 amino acids.

Embodiment 129 comprises an isolated polypeptide or polypeptide complex of any one of embodiments 101-125, wherein L₃ₐ is a peptide sequence having at least 18 amino acids.

Embodiment 130 comprises an isolated polypeptide or polypeptide complex of any one of embodiments 101-125, wherein L₃ₐ is a peptide sequence having at least 26 amino acids.

Embodiment 131 comprises an isolated polypeptide or polypeptide complex of any one of embodiments 101-130, wherein L₃ₐ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1.

Embodiment 132 comprises an isolated polypeptide or polypeptide complex of any one of embodiments 101-131, wherein L₃ₐ comprises an amino acid sequence according to GGGGSGGGS (SEQ ID NO: 19).

### Examples

### Example 1: Serum Albumin Binding ELISA

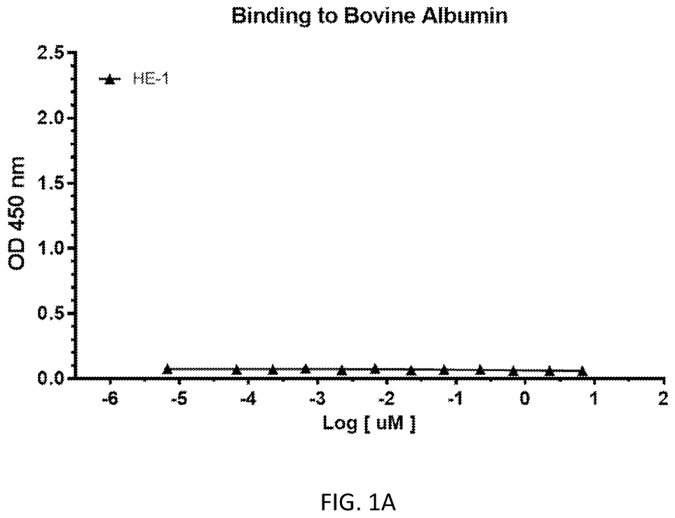

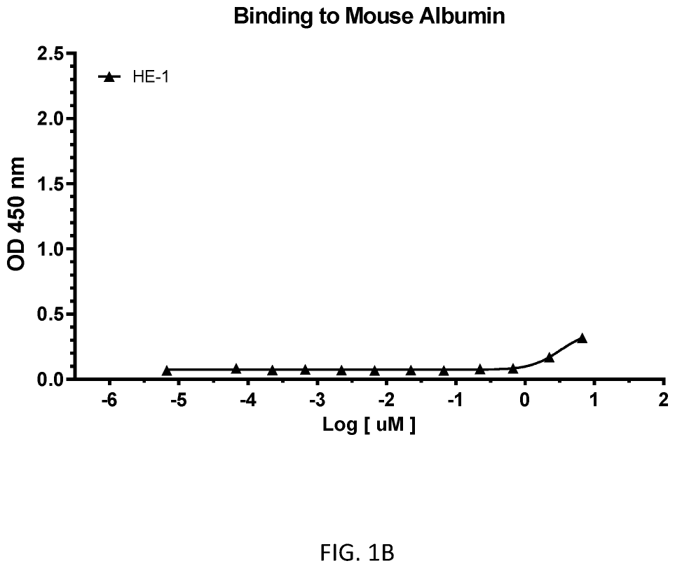

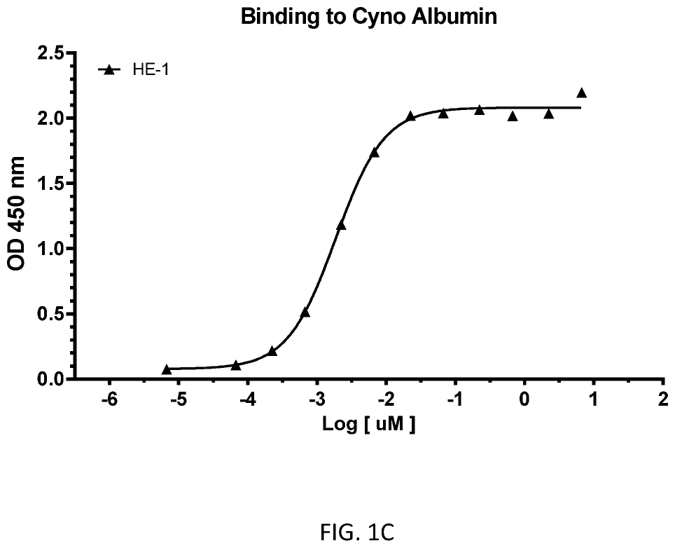

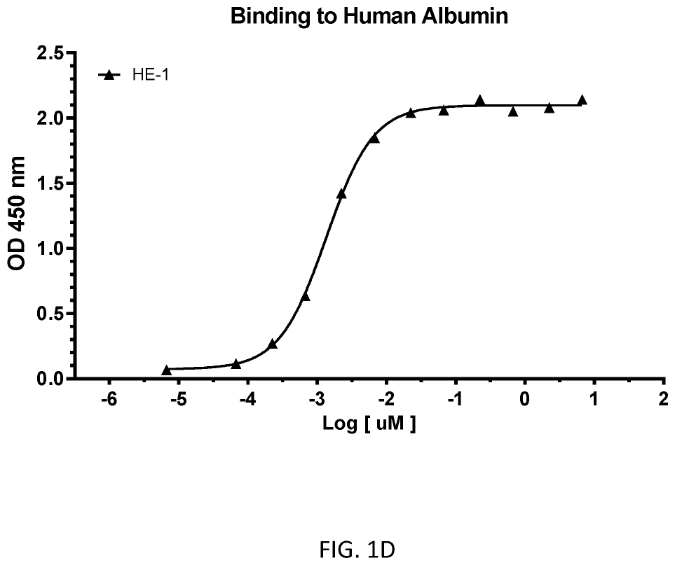

Single domain antibodies were evaluated for their ability to bind serum albumin from mouse, bovine, cynomolgus monkey, and human by enzyme linked immunosorbent assays (ELISAs). Briefly, single domain antibodies were titrated into albumin coated ELISA plates. After washing, bound single domain antibodies were detected using a secondary horse radish peroxidase antibody conjugate. After washing again, plates were developed using standard ELISA techniques and stopped using acid. The concentration of single domain antibody required to achieve 50% maximal signal or EC50 was calculated using Graphpad prism. Data is shown in FIG. 1A-FIG. 1D.

### Example 2: pH Dependent Kinetic Binding of Human Serum Albumin

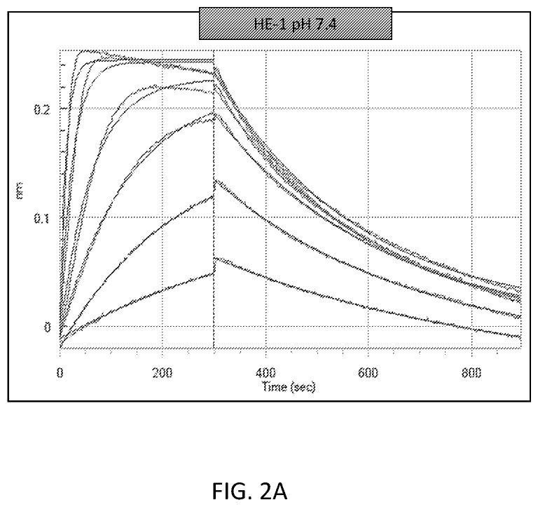

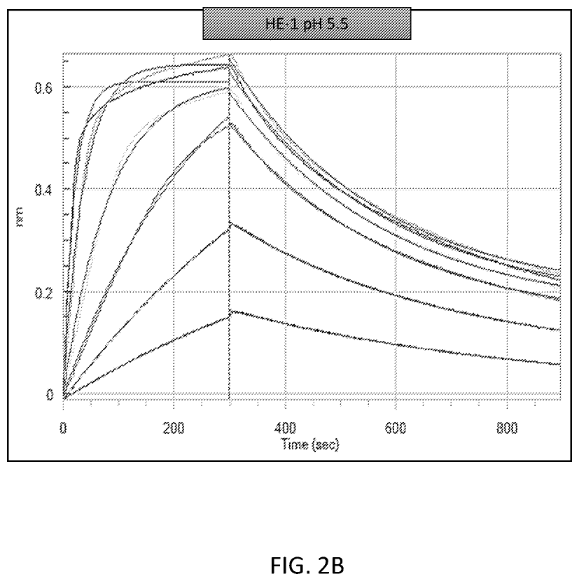

Single domain antibody binding kinetics against human serum albumin were evaluated using an Octet RED96 instrument. Briefly, biotinylated human serum albumin was captured on streptavidin biosensors, quenched with biocytin, and baselined in buffer. Single domain antibodies titrated in buffer at neutral or acidic pH were then associated onto the albumin loaded biosensors. After association, biosensors were transferred to neutral or acidic pH buffer and the single domain antibody dissociation was measured. The association and dissociation kinetics were measured in real time and fit to a one to one binding model. The on rate, off rate, and affinity (KD) were then compared across different single domain antibodies. Data is shown in FIG. 2A-2B.

TABLE 2
Summary of settings provided in FIG. 2A.

| Step | Time | pH |
|---|---|---|
| Baseline: Octet buffer | 60 sec | pH 7.4 |
| Load: 30 nM Human albumin-biotin | 300 sec | pH 7.4 |
| (2.106 ug/mL) | | |
| Biocytin quench (100 uM) | 300 sec | pH 7.4 |
| Baseline: Octet buffer | 300 sec | pH 7.4 |
| Association in octet buffer | 300 sec | pH 7.4 |
| HE-1 anti-albumin | | |
| 100 nM, 50 nM, 25 nM, | | |
| 12.5 nM, 6.25 nM, 3.125 nM | | |
| Dissociation: Octet Buffer | 600 sec | pH 7.4 |

TABLE 3
Summary of plots provided in FIG. 2A.

| | Loading | | | |
|---|---|---|---|---|
| Sample ID | Sample ID | KD (M) | kon(1/Ms) | kdis(1/s) |
| HE-1 anti- | HSA-biotin | 5.98E−09 | 5.71E+05 | 3.41E−03 |
| albumin | | | | |

TABLE 4
Summary of settings provided in FIG. 2B.

| Step | Time | pH |
|---|---|---|
| Baseline: Octet buffer | 60 sec | pH 7.4 |
| Load: 30 nM Human albumin-biotin (2.106 ug/mL) | 300 sec | pH 7.4 |
| Biocytin quench (100 uM) | 300 sec | pH 7.4 |
| Baseline: Octet buffer | 300 sec | pH 5.5 |
| Association in octet buffer | 300 sec | pH 5.5 |
| HE-1 anti-albumin | | |
| 100 nM, 50 nM, 25 nM, 12.5 nM, 6.25 nM, | | |
| 3.125 nM | | |
| Dissociation: Octet Buffer | 600 sec | pH 5.5 |

TABLE 5
Summary of plots provided in FIG. 2B.

| Sample ID | Loading Sample ID | KD (M) | kon(1/Ms) | kdis(1/s) |
|---|---|---|---|---|
| HE-1 anti- | HSA-biotin | 4.19E−09 | 4.44E+05 | 1.86E−03 |
| albumin | | | | |

### Example 3: pH Dependent Binding to Cyno and Human Albumin by ELISA

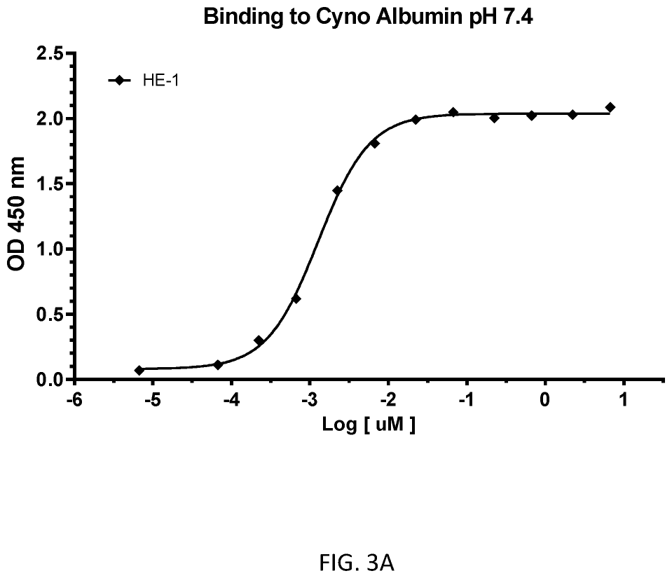

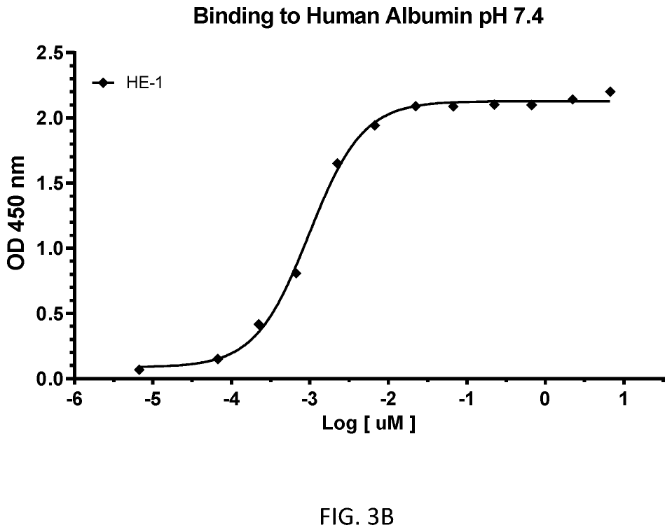

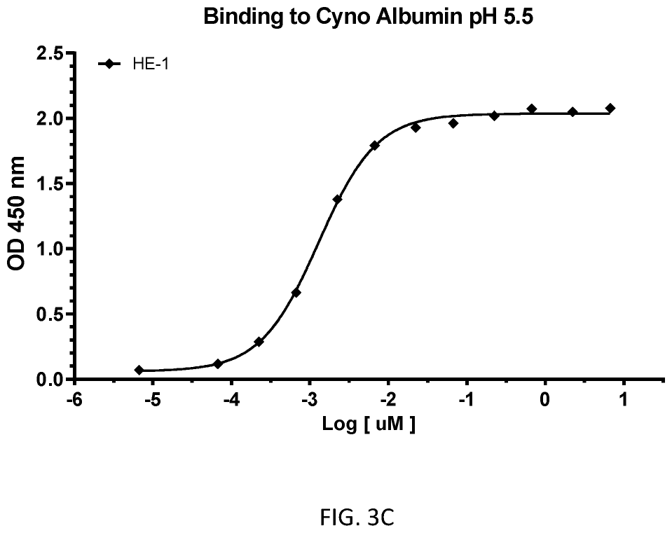

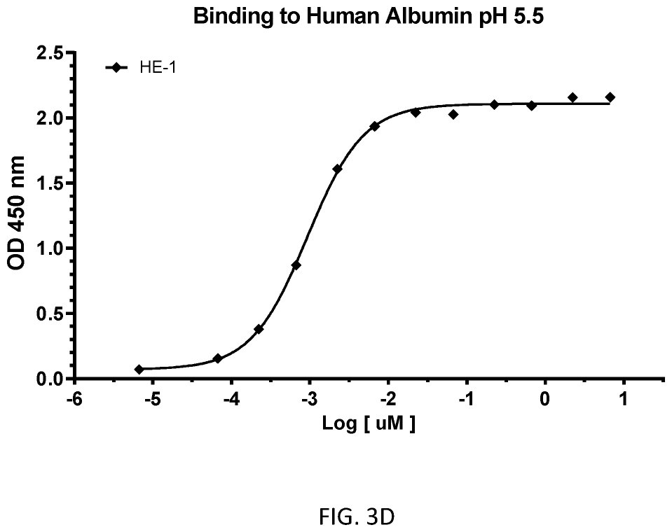

Single domain antibodies were evaluated for their ability to bind serum albumin from cynomolgus monkey or human by enzyme linked immunosorbent assays (ELISAs) at neutral and acidic pH. Briefly, single domain antibodies were titrated into neutral or acidic buffer onto albumin coated ELISA plates. After washing, bound single domain antibodies were detected using a secondary horse radish peroxidase antibody conjugate. After washing again, plates were developed using standard ELISA techniques and stopped using acid. The concentration of single domain antibody required to achieve 50% maximal signal or EC50 was calculated using Graphpad prism. Single domain antibody affinity at neutral and acidic pH were then compared. Data is shown in FIG. 3A-FIG. 3D.

TABLE 6
Summary of plots provided in FIG. 3A-FIG. 3D.

| | | |
|---|---|---|
| HE-1 anti-albumin | pH 7.4 | pH 5.5 |
| Cyno | 1.3 nM | 1.3 nM |
| Human | 1.0 nM | 0.9 nM |

### Example 4: Single Domain Antibody and FcRn Kinetic Binding Competition to Human Albumin

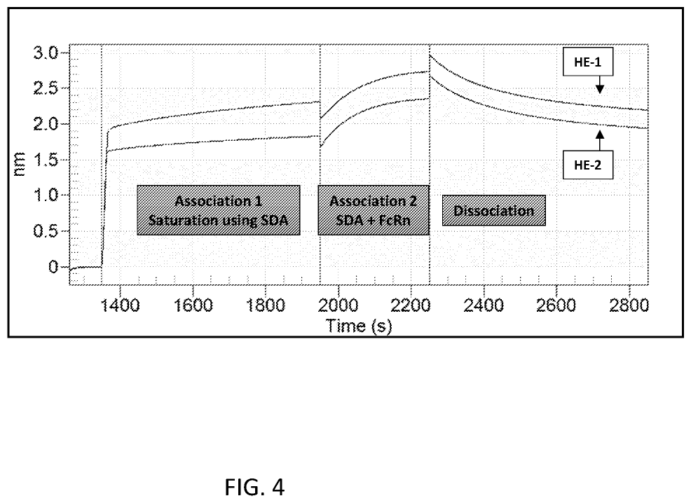

Single domain antibodies were evaluated for their ability to bind human serum albumin without blocking FcRn recognition. Briefly, biotinylated human serum albumin was loaded on streptavidin biosensors, quenched in biocytin, and baselined in acidic buffer. Single domain antibodies were then associated onto the sensor until saturation occurred (Association 1). Sensors were then transferred to the same concentration of single domain antibodies now also containing FcRn (association 2). An increase in signal during the second association step implies that FcRn can still bind human albumin in the context of albumin already saturated with bound single domain antibodies. Hence, association 2 tests whether the single domain antibodies compete with FcRn for albumin binding sites. Lastly, biosensors were transferred to buffer and dissociation was observed. A positive control single domain antibody known to lack FcRn competitive binding of albumin was used for comparisons. Data is shown in FIG. 4.

### Example 5: Polypeptide Sequences for In Vivo Half-Life Extension

Polypeptide complexes PC1, PC2, PC3, and PC4 have light chain (LC) and heavy chain (HC) amino acid sequences disclosed in Table 7. The HE-1 sequence (SEQ ID NO: 4) is incorporated into PC2 LC and in PC4 HC. PC2 and PC4 which have the incorporated HE-1 sequence are compared against PC1 and PC3, respectively, to show the effect of HE-1 on in vivo half-life extension in cynomolgus monkeys. SEQ ID NO:7 and SEQ ID NO:8 are the respective LC and HC amino acid sequences of PC1. SEQ ID NO:9 and SEQ ID NO: 10 are the respective LC and HC amino acid sequences of PC2. SEQ ID NO:11 and SEQ ID NO: 12 are the respective LC and HC amino acid sequences of PC3. SEQ ID NO: 13 and SEQ ID NO: 14 are the respective LC and HC amino acid sequences of PC4. PC1 and PC2 include PSMA and CD3 targeting regions, and PC3 and PC4 include TROP2 and CD3 targeting regions. PC2 includes a single mask, and PC4 includes a double mask. PC2 has a configuration according to Configuration 8, and PC4 has a configuration according to Configuration 5.

TABLE 7
Polypeptide complex sequences

| Construct | Amino Acid Sequence | SEQ ID |
|---|---|---|
| Description | (N to C) | NO: |
| PC1:LC | EVQLVESGGGLVQPGGSLKLSCAASGFTFNKYAMNW | 7 |
| PSMA | VRQAPGKGLEWVARIRSKYNNYATYYADSVKDRFTI | |
| | SRDDSKNTAYLQMNNLKTEDTAVYYCVRHGNFGNS | |
| | YISYWAYWGQGTLVTVSSGGGGSGGGGSGGGGSQTV | |
| | VTQEPSLTVSPGGTVTLTCGSSTGAVTSGNYPNWVQQ | |
| | KPGQAPRGLIGGTKFLAPGTPARFSGSLLGGKAALTLS | |
| | GVQPEDEAEYYCVLWYSNRWVFGGGTKLTVLGGGG | |
| | SDIQMTQSPSSLSASVGDRVTITCRASQGISNYLAWYQ | |
| | QKTGKVPKFLIYEASTLQSGVPSRFSGGGSGTDFTLTIS | |
| | SLQPEDVATYYCQNYNSAPFTFGPGTKVDIKRTVAAP | |
| | SVFIFPPSDEQLKSGTASVVCLLNNFYPREAKVQWKV | |
| | DNALQSGNSQESVTEQDSKDSTYSLSSTLTLSKADYE | |
| | KHKVYACEVTHQGLSSPVTKSFNRGEC | |
| PC1:HC | QVQLVESGGGVVQPGRSLRLSCAASGFAFSRYGMHW | 8 |
| PSMA | VRQAPGKGLEWVAVIWYDGSNKYYADSVKGRFTISR | |
| | DNSKNTQYLQMNSLRAEDTAVYYCARGGDFLYYYY | |
| | YGMDVWGQGTTVTVSSASTKGPSVFPLAPSSKSTSGG | |
| | TAALGCLVKDYFPEPVTVSWNSGALTSGVHTFPAVLQ | |
| | SSGLYSLSSVVTVPSSSLGTQTYICNVNHKPSNTKVDK | |
| | KVEPKSC | |
| PC2:LC | EVQLVESGGGLVQPGGSLRLSCAASGSTFYTAVMGW | 9 |
| PSMA | VRQAPGKGLEWVAAIRWTALTTSYADSVKGRFTISRD | |
| | GAKTTLYLQMNSLRPEDTAVYYCAARGTLGLFTTAD | |
| | SYDYWGQGTLVTVSSGGGGSGGGSGGVYCGPEFDES | |
| | VGCMGGGGSGGGLSGRSDAGSPLGLAGSGGGSEVQL | |
| | VESGGGLVQPGGSLKLSCAASGFTFNKYAMNWVRQA | |
| | PGKGLEWVARIRSKYNNYATYYADSVKDRFTISRDDS | |
| | KNTAYLQMNNLKTEDTAVYYCVRHGNFGNSYISYW | |
| | AYWGQGTLVTVSSGGGGSGGGGSGGGGSQTVVTQEP | |
| | SLTVSPGGTVTLTCGSSTGAVTSGNYPNWVQQKPGQ | |
| | APRGLIGGTKFLAPGTPARFSGSLLGGKAALTLSGVQP | |
| | EDEAEYYCVLWYSNRWVFGGGTKLTVLGGGGSDIQ | |
| | MTQSPSSLSASVGDRVTITCRASQGISNYLAWYQQKT | |
| | GKVPKFLIYEASTLQSGVPSRFSGGGSGTDFTLTISSLQ | |
| | PEDVATYYCQNYNSAPFTFGPGTKVDIKRTVAAPSVFI | |
| | FPPSDEQLKSGTASVVCLLNNFYPREAKVQWKVDNA | |
| | LQSGNSQESVTEQDSKDSTYSLSSTLTLSKADYEKHK | |
| | VYACEVTHQGLSSPVTKSFNRGEC | |
| PC2:HC | QVQLVESGGGVVQPGRSLRLSCAASGFAFSRYGMHW | 10 |
| PSMA | VRQAPGKGLEWVAVIWYDGSNKYYADSVKGRFTISR | |
| | DNSKNTQYLQMNSLRAEDTAVYYCARGGDFLYYYY | |
| | YGMDVWGQGTTVTVSSASTKGPSVFPLAPSSKSTSGG | |
| | TAALGCLVKDYFPEPVTVSWNSGALTSGVHTFPAVLQ | |
| | SSGLYSLSSVVTVPSSSLGTQTYICNVNHKPSNTKVDK | |
| | KVEPKSC | |
| PC3:LC | DIQLTQSPSSLSASVGDRVSITCKASQDVSIAVAWYQQ | 11 |
| TROP2 | KPGKAPKLLIYSASYRYTGVPDRFSGSGSGTDFTLTISS | |
| | LQPEDFAVYYCQQHYITPLTFGAGTKVEIKRTVAAPS | |
| | VFIFPPSDEQLKSGTASVVCLLNNFYPREAKVQWKVD | |
| | NALQSGNSQESVTEQDSKDSTYSLSSTLTLSKADYEK | |
| | HKVYACEVTHQGLSSPVTKSFNRGEC | |
| PC3:HC | QTVVTQEPSLTVSPGGTVTLTCRSSTGAVTTSNYANW | 12 |
| TROP2 | VQQKPGQAPRGLIGGTNKRAPGTPARFSGSLLGGKAA | |
| | LTLSGVQPEDEAEYYCALWYSNLWVFGGGTKLTVLG | |
| | GGGSGGGGSGGGGSEVQLVESGGGLVQPGGSLKLSC | |
| | AASGFTFNTYAMNWVRQAPGKGLEWVARIRSKYNN | |
| | YATYYADSVKDRFTISRDDSKNTAYLQMNNLKTEDT | |
| | AVYYCVRHGNFGNSYVSWFAYWGQGTLVTVSSGGG | |
| | GSQVQLQQSGSELKKPGASVKVSCKASGYTFTNYGM | |
| | NWVKQAPGQGLKWMGWINTYTGEPTYTDDFKGRFA | |
| | FSLDTSVSTAYLQISSLKADDTAVYFCARGGFGSSYW | |
| | YFDVWGQGSLVTVSSASTKGPSVFPLAPSSKSTSGGT | |
| | AALGCLVKDYFPEPVTVSWNSGALTSGVHTFPAVLQS | |
| | SGLYSLSSVVTVPSSSLGTQTYICNVNHKPSNTKVDKK | |
| | VEPKSC | |
| PC4:LC | GGVDFCKIYSWPVCHQGGGGSGGLSGRSDAGSPLGL | 13 |
| TROP2 | AGSGGSDIQLTQSPSSLSASVGDRVSITCKASQDVSIAV | |
| | AWYQQKPGKAPKLLIYSASYRYTGVPDRFSGSGSGTD | |
| | FTLTISSLQPEDFAVYYCQQHYITPLTFGAGTKVEIKRT | |
| | VAAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGEC | |
| PC4:HC | EVQLVESGGGLVQPGGSLRLSCAASGSTFYTAVMGW | 14 |
| TROP2 | VRQAPGKGLEWVAAIRWTALTTSYADSVKGRFTISRD | |
| | GAKTTLYLQMNSLRPEDTAVYYCAARGTLGLFTTAD | |
| | SYDYWGQGTLVTVSSGGGGSGGGSGGVYCGPEFDES | |
| | VGCMGGGGSGGGLSGRSDAGSPLGLAGSGGGSEVQL | |
| | VESGGGLVQPGGSLKLSCAASGFTFNKYAMNWVRQA | |
| | PGKGLEWVARIRSKYNNYATYYADSVKDRFTISRDDS | |
| | KNTAYLQMNNLKTEDTAVYYCVRHGNFGNSYISYW | |
| | AYWGQGTLVTVSSGGGGSGGGGSGGGGSQTVVTQEP | |
| | SLTVSPGGTVTLTCGSSTGAVTSGNYPNWVQQKPGQ | |
| | APRGLIGGTKFLAPGTPARFSGSLLGGKAALTLSGVQP | |
| | EDEAEYYCVLWYSNRWVFGGGTKLTVLGGGGSQVQ | |
| | LQQSGSELKKPGASVKVSCKASGYTFTNYGMNWVKQ | |
| | APGQGLKWMGWINTYTGEPTYTDDFKGRFAFSLDTS | |
| | VSTAYLQISSLKADDTAVYFCARGGFGSSYWYFDVW | |
| | GQGSLVTVSSASTKGPSVFPLAPSSKSTSGGTAALGCL | |
| | VKDYFPEPVTVSWNSGALTSGVHTFPAVLQSSGLYSL | |
| | SSVVTVPSSSLGTQTYICNVNHKPSNTKVDKKVEPKSC | |

### Example 6: PSMA and CD3e Binding by Polypeptide Complexes (PC1 and PC2)

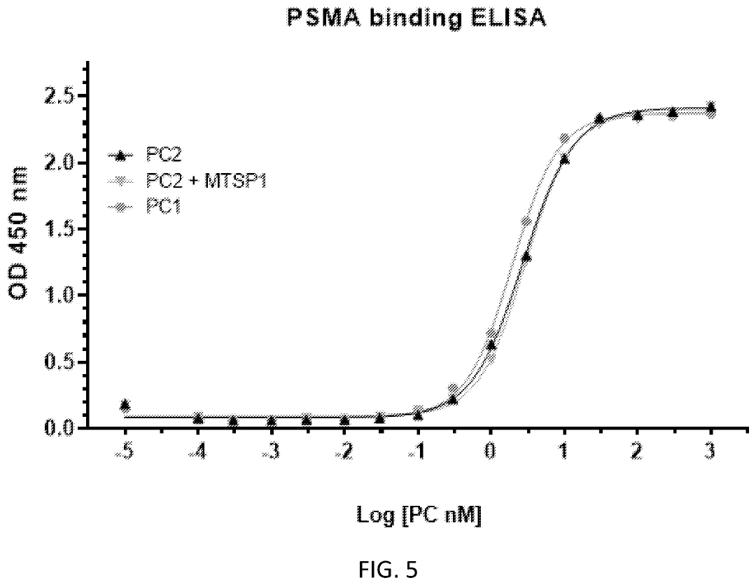

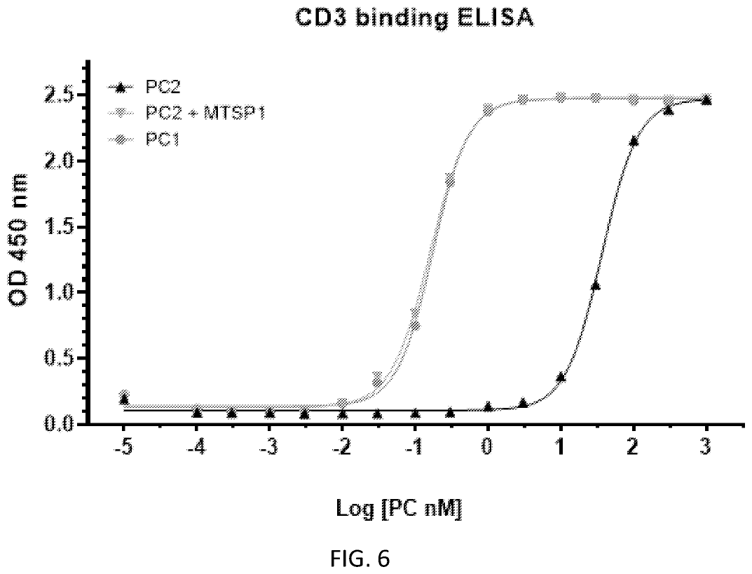

Polypeptide complexes PC1 and PC2 were evaluated for binding to PSMA and CD3e using ELISA. Binding studies with PC2 were also performed in the presence of matriptase enzyme (MTSP1) for cleavage of the mask. Binding data for PSMA and CD3e are shown in FIGS. 5 and 6, respectively.

### Example 7: Polypeptide Complex Mediated Killing of 22RV1 Tumor Cells in the Presence of CD8+ T Cells

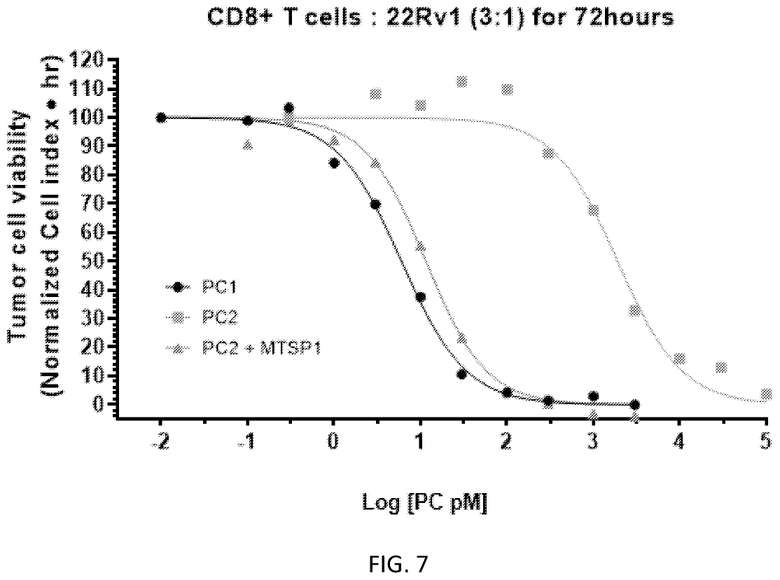

Polypeptide complexes PC1 and PC2 were evaluated in a functional in vitro tumor cell killing assay using the PSMA positive tumor cell lines 22Rv1. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed, the impedance decreased. 10,000 tumor cells were added per well and allowed to adhere overnight on a 96 well E-Plate. The following day, polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex, where the concentration required to reduce the tumor growth by 50% (IC50) was calculated using Graphpad Prism software. FIG. 7 shows representative cytotoxicity data for 22Rv1 treated with PC1, PC2, and PC2 cleaved with MTSP1. The data is summarized in Table 8, and shows that PC1 is more than 300 times more cytotoxic to the 22Rv1 cells than PC2. Unmasking of PC2 by enzymatic cleavage increased the cytotoxicity by more than 150 fold.

TABLE 8
22Rv1 cell viability

| 22Rv1 IC50 | | | |
|---|---|---|---|
| (pM) | PC1 | PC2 | PC2 + MTSP1 |
| 72 hours | 6.01 | 1,902 | 11.5 |

### Example 8: Pharmacokinetics of PC1 and PC2 in Cynomolgus Monkey

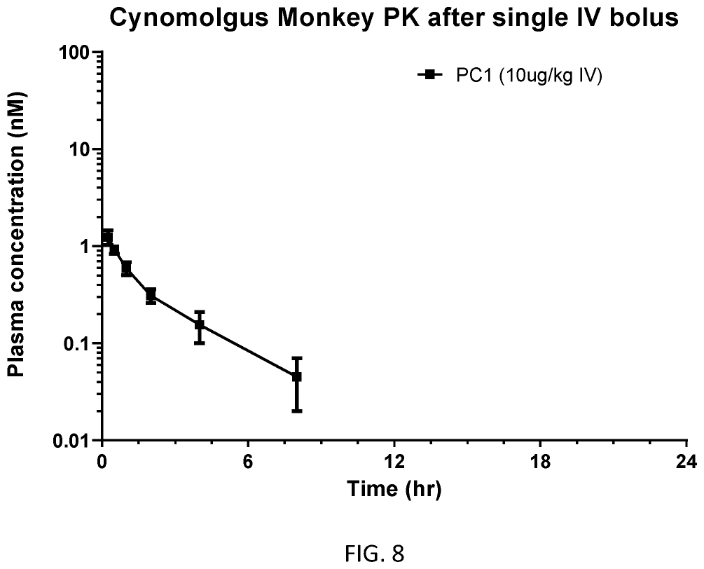

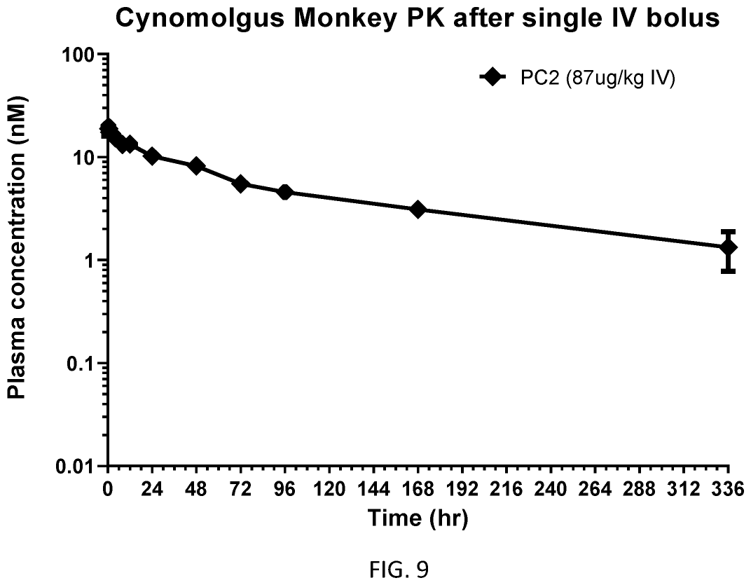

Pharmacokinetics and exploratory safety of polypeptide molecules PC1 and PC2 were evaluated in cynomolgus monkeys. Briefly, cynomolgus monkeys of approximately 3 kilogram (kg) bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentrations of polypeptide molecules in plasma were measured via standard ELISA techniques relative to a reference standard diluted in control cynomolgus plasma. Plasma concentration curves were fit to a standard two phase exponential equation representing distribution and elimination phases. Fitting of pharmacokinetics enabled the calculation of C_{MAX}, half-life (t_{1/2}), volume of distribution (Vd), clearance (CL), and 7 day area under the curve (AUC) as shown in Table 9 for PC1 and in Table 10 for PC2. Pharmacokinetic data for PC1 (10 μg/kg IV) is shown in FIG. 8. Pharmacokinetic data for PC2 (87 μg/kg IV) is shown in FIG. 9. The measured pharmacokinetics in cynomolgus monkeys support once weekly dosing in humans.

TABLE 9
PC1 pharmacokinetic data

| | PC1 10 μg/kg | Units |
|---|---|---|
| C_{MAX} | 1.69 | nM |
| t_{1/2} | 2.17 | Hr |
| Vd | 0.23 | L |
| VSS | 0.67 | L |
| CL | 24.49 | mL/hr/kg |
| BW | 3.00 | Kg |
| 7day | 141 | nM · min |
| AUC | | |

TABLE 10
PC2 pharmacokinetic data

| | PC2 87 μg/kg | Units |
|---|---|---|
| C_{MAX} | 17.90 | nM |
| t_{1/2} | 118.99 | hr |
| Vd | 0.18 | L |
| VSS | 0.35 | L |
| CL | 0.34 | mL/hr/kg |
| BW | 3.00 | kg |
| 7day | 63,731 | nM · min |
| AUC | | |

### Example 9: TROP2 and CD3e Binding by Polypeptide Complexes (PC1 and PC4)

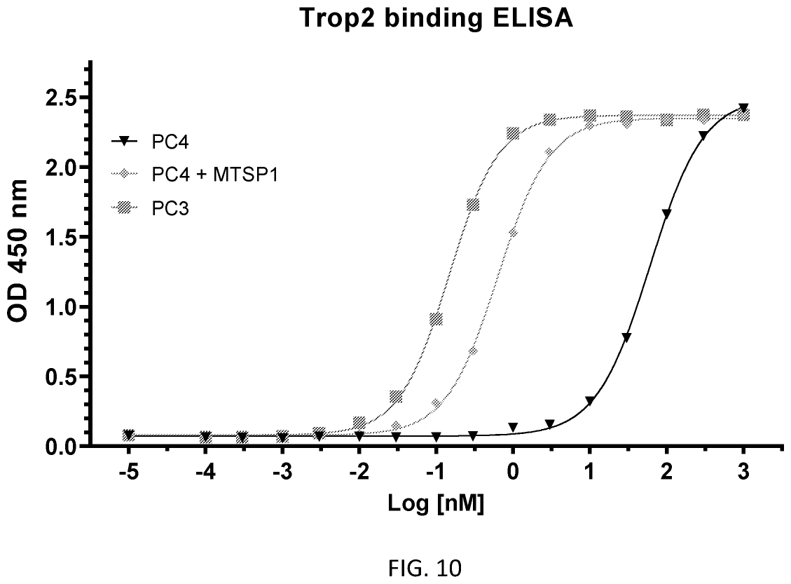

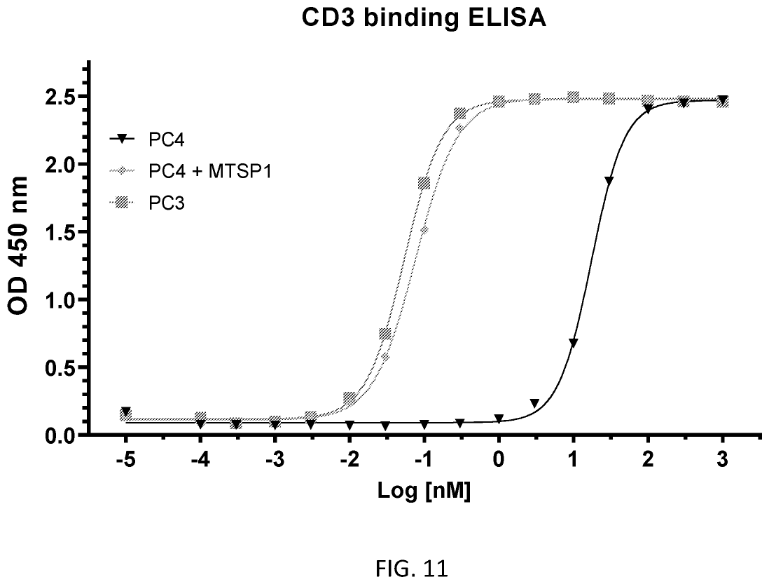

Polypeptide complexes PC1 and PC4 were evaluated for binding to TROP2 and CD3e using ELISA. Binding studies with PC4 were also performed in the presence of matripase (MTSP1) for mask cleavage. Binding data for TROP2 and CD3e are shown in FIGS. 10 and 11, respectively.

### Example 10: Polypeptide Complex Mediated Killing of H292 Cells in the Presence of CD8+ T Cells

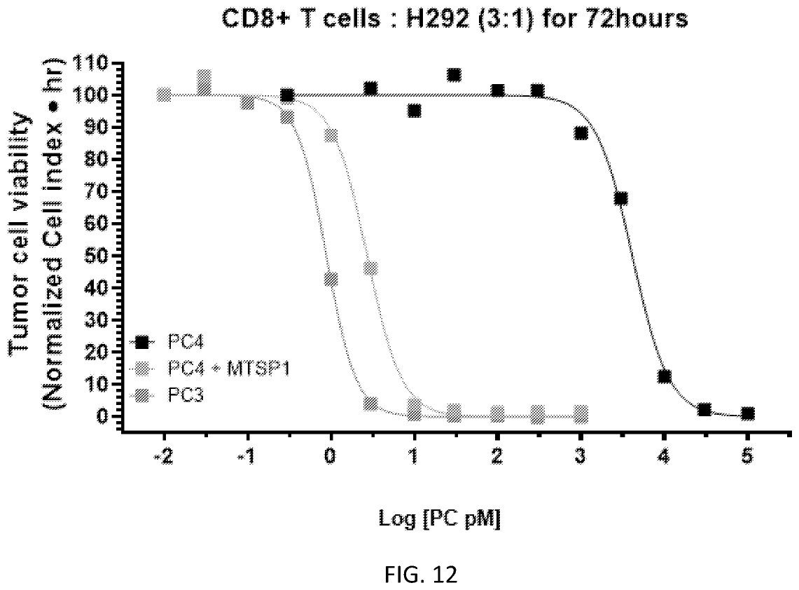

Polypeptide complexes PC3 and PC4 were evaluated in a functional in vitro tumor cell killing assay using the TROP2 positive tumor cell lines HCT116, NCI-H292, and MDAMB231. Tumor cell killing was measuring using an xCelligence real time cell analyzer from Agilent that relies on sensor impedance measurements (cell index) that increased as tumor cell lines adhere, spread, and expand on the surfaces of the sensor. Likewise, as the tumor cells were killed, the impedance decreased. 10,000 tumor cells were added per well and allowed to adhere overnight on a 96 well E-Plate. The following day, polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex where the concentration required to reduce the tumor growth 50% (IC50) was calculated using Graphpad Prism software. FIG. 12 shows representative cytotoxicity data for H292 cells treated with PC3, PC4, and PC4 cleaved with MTSP1. The data is summarized in Table 11. Referring to Table 11, PC3 is more than 4,000 times more cytotoxic to the H292 cells than PC4. Cleavage of PC4 with MTSP-1 dramatically increased the cytotoxicity of the polypeptide complex.

TABLE 11
H292 cell viability

| H292 IC50 (pM) | PC3 | PC4 | PC4 + MTSP1 |
|---|---|---|---|
| 72 hours | 0.88 | 4,118 | 2.72 |

### Example 11: Pharmacokinetics of PC3 and PC4 in Cynomolgus Monkey

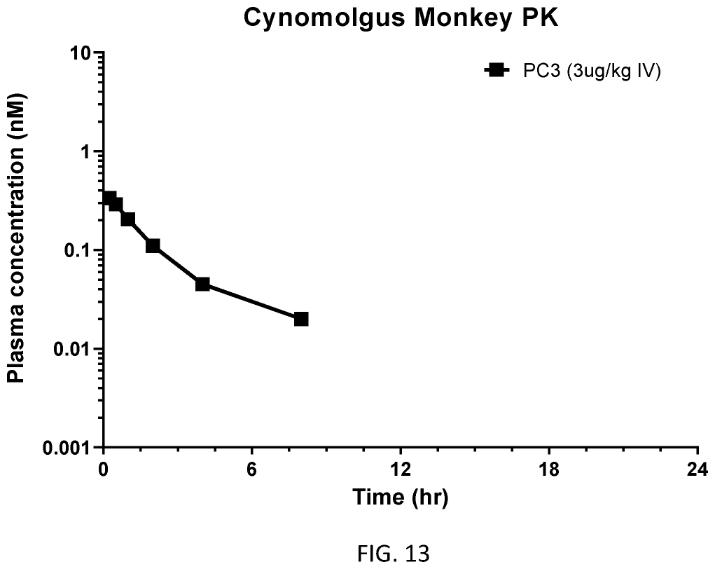

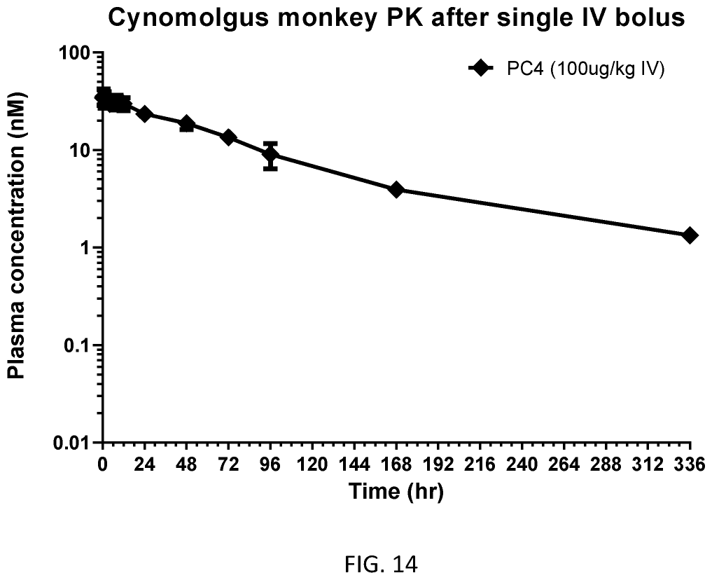

Pharmacokinetics and exploratory safety of polypeptide molecules PC3 and PC4 were evaluated in cynomolgus monkeys. Briefly, cynomolgus monkeys of approximately 3 kilogram (kg) bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentrations of polypeptide molecules in plasma were measured via standard ELISA techniques relative to a reference standard diluted in control cynomolgus plasma. Plasma concentration curves were fit to a standard two phase exponential equation representing distribution and elimination phases. Fitting of pharmacokinetics enabled the calculation of C_{MAX}, half-life (t_{1/2}), volume of distribution (Vd), clearance (CL), and 7 day area under the curve (AUC) as shown in Table 12 for PC3 and in Table 13 for PC4. Pharmacokinetic data for PC3 (3 μg/kg IV) is shown in FIG. 13. Pharmacokinetic data for PC4 (1000 μg/kg IV) is shown in FIG. 14. The measured pharmacokinetics in cynomolgus monkeys support once weekly dosing in humans.

TABLE 12
PC3 pharmacokinetic data

| | PC3 3 μg/kg | Units |
|---|---|---|
| C_{MAX} | 0.40 | nM |
| t_{1/2} | 1.02 | hr |
| Vd | 0.30 | L |
| VSS | 0.30 | L |
| CL | 67.31 | mL/hr/kg |
| BW | 3.00 | kg |
| 7day | 49 | nM · min |
| AUC | | |

TABLE 13
PC4 pharmacokinetic data

| | PC4 100 μg/kg | Units |
|---|---|---|
| C_{MAX} | 34.32 | nM |
| t_{1/2} | 90.16 | hr |
| Vd | 0.09 | L |
| VSS | 0.19 | L |
| CL | 0.23 | mL/hr/kg |
| BW | 3.00 | kg |
| 7day | 142,182 | nM · min |
| AUC | | |

What is claimed is:

1. An isolated polypeptide or polypeptide complex comprising a half-life extending antibody (H₁) or antibody fragment that comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3; and wherein the CDRs comprise from 0-2 amino acid modifications in at least one of the HC-CDR1, HC-CDR2, or HC-CDR3.

2. The isolated polypeptide or polypeptide complex of claim 1, wherein H₁ comprises complementarity determining regions (CDRs): HC-CDR1, HC-CDR2, and HC-CDR3, wherein the HC-CDR1, the HC-CDR2, and the HC-CDR3 of H₁ comprise: HC-CDR1: SEQ ID NO: 1, HC-CDR2: SEQ ID NO: 2, and HC-CDR3: SEQ ID NO: 3.

3. The isolated polypeptide or polypeptide complex of claim 1, wherein the antibody or antibody fragment of H₁ comprises a single domain antibody, a single chain variable fragment, a Fab, or Fab′.

4. The isolated polypeptide or polypeptide complex of claim 3, wherein the antibody or antibody fragment of H₁ comprises the single domain antibody.

5. The isolated polypeptide or polypeptide complex of claim 1, wherein H₁ comprises an amino acid sequence with at least 90% sequence identity to SEQ ID NO: 4.

6. The isolated polypeptide or polypeptide complex of claim 1, wherein H₁ comprises an amino acid sequence with at least 95% sequence identity to SEQ ID NO: 4.

7. The isolated polypeptide or polypeptide complex of claim 1, wherein H₁ comprises an amino acid sequence with at least 99% sequence identity to SEQ ID NO: 4.

8. The isolated polypeptide or polypeptide complex of claim 1, wherein H₁ comprises the amino acid sequence according to SEQ ID NO: 4.

9. The isolated polypeptide or polypeptide complex of claim 1, wherein H₁ comprises an amino acid sequence of at least 110 consecutive amino acid residues of SEQ ID NO: 4.

10. The isolated polypeptide or polypeptide complex of claim 1, wherein H₁ comprises an amino acid sequence of at least 120 consecutive amino acid residues of SEQ ID NO: 4.

11. The isolated polypeptide or polypeptide complex of claim 1, wherein H₁ comprises an amino acid sequence of at least 110 consecutive amino acid residues of SEQ ID NO: 4 and has at least 95% sequence identity to the at least 110 consecutive amino acid residues of SEQ ID NO: 4.

12. The isolated polypeptide or polypeptide complex of claim 1, wherein H₁ comprises an amino acid sequence of at least 120 consecutive amino acid residues of SEQ ID NO: 4 and has at least 95% sequence identity to the at least 120 consecutive amino acid residues of SEQ ID NO: 4.

13. The isolated polypeptide or polypeptide complex of claim 1, wherein H₁ is linked to a peptide (P₁) that impairs binding of a first antigen recognizing molecule (A₁) to a first target antigen through a cleavable linker (L₁) that is a substrate for a tumor specific protease in a configuration according to Formula I:
A₁-L₁-P₁-H₁ (Formula I).

14. The isolated polypeptide or polypeptide complex of claim 13, wherein A₁ is further linked to a second antigen recognizing molecule (A₂).

15. The isolated polypeptide or polypeptide complex of claim 14, wherein the polypeptide or polypeptide complex is according to Formula Ia:
P₂-L₂-A₂-A₁-L₁-P₁-H₁ (Formula Ia)
wherein P₂ comprises a peptide that impairs binding of A₂ to a second target antigen; and L₂ comprises a second cleavable linker that connects A₂ to P₂ and is a substrate for a tumor specific protease.

16. The isolated polypeptide or polypeptide complex of claim 1, wherein H₁ comprises a linking moiety (L₃) that connects H₁ to P₁.

17. The isolated polypeptide or polypeptide complex of claim 16, wherein L₃ is a peptide sequence having at least 5 to no more than 50 amino acids.

18. The isolated polypeptide or polypeptide complex of claim 16, wherein L₃ is a peptide sequence having at least 10 to no more than 30 amino acids.

19. The isolated polypeptide or polypeptide complex of claim 16, wherein L₃ is a peptide sequence having at least 10 amino acids.

20. The isolated polypeptide or polypeptide complex of claim 16, wherein L₃ is a peptide sequence having at least 18 amino acids.

21. The isolated polypeptide of claim 16, wherein L₃ is a peptide sequence having at least 26 amino acids.

22. The isolated polypeptide or polypeptide complex of claim 16, wherein L₃ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1.

23. The isolated polypeptide of claim 22, wherein L₃ comprises an amino acid sequence GGGGSGGGS (SEQ ID NO: 19).

24. The isolated polypeptide or polypeptide complex of claim 14, wherein A₁ comprises an antibody or antibody fragment.

25. The isolated polypeptide or polypeptide complex of claim 24, wherein A₁ comprises an antibody or antibody fragment that is human or humanized.

26. The isolated polypeptide or polypeptide complex of claim 24, wherein L₁ is bound to a N-terminus of the antibody or antibody fragment of A₁.

27. The isolated polypeptide or polypeptide complex of claim 24, wherein A₂ is bound to N-terminus of the antibody or antibody fragment of A₁.

28. The isolated polypeptide or polypeptide complex of claim 24, wherein L₁ is bound to a C-terminus of the antibody or antibody fragment of A₁.

29. The isolated polypeptide or polypeptide complex of claim 24, wherein A₂ is bound to a C-terminus of the antibody or antibody fragment of A₁.

30. The isolated polypeptide or polypeptide complex of claim 24, wherein the antibody or antibody fragment of A₁ comprises a single chain variable fragment, a single domain antibody, or a Fab fragment.

31. The isolated polypeptide or polypeptide complex of claim 30, wherein A₁ is the single chain variable fragment (scFv).

32. The isolated polypeptide or polypeptide complex of claim 31, wherein the scFv comprises a scFv heavy chain polypeptide and a scFv light chain polypeptide.

33. The isolated polypeptide or polypeptide complex of claim 30, wherein A₁ is the single domain antibody.

34. The isolated polypeptide or polypeptide complex of claim 30, wherein the antibody or antibody fragment thereof of A₁ comprises a single chain variable fragment (scFv), a heavy chain variable domain (VH domain), a light chain variable domain (VL domain), or a variable domain (VHH) of a camelid derived single domain antibody.

35. The isolated polypeptide or polypeptide complex of claim 13, wherein the first target antigen comprises an effector cell antigen.

36. The isolated polypeptide or polypeptide complex of claim 13, wherein the first target antigen is CD3.

37. The isolated polypeptide or polypeptide complex of claim 13, wherein A₁ comprises an anti-CD3e single chain variable fragment.

38. The isolated polypeptide or polypeptide complex of claim 13, wherein A₁ comprises an anti-CD3e single chain variable fragment that has a KD binding of 1 μM or less to CD3 on CD3 expressing cells.

39. The isolated polypeptide or polypeptide complex of claim 13, wherein A₁ comprises a variable light chain and variable heavy chain each of which is capable of specifically binding to human CD3.

40. The isolated polypeptide or polypeptide complex of claim 13, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

41. The isolated polypeptide or polypeptide complex of claim 13, wherein the polypeptide or polypeptide complex of formula I binds to an effector cell when L₁ is cleaved by the tumor specific protease.

42. The isolated polypeptide or polypeptide complex of claim 13, wherein the polypeptide or polypeptide complex of formula I binds to an effector cell when L₁ is cleaved by the tumor specific protease and A₁ binds to the effector cell.

43. The isolated polypeptide or polypeptide complex of claim 42, wherein the effector cell is a T cell.

44. The isolated polypeptide or polypeptide complex of claim 43, wherein A₁ binds to a polypeptide that is part of a TCR-CD3 complex on the effector cell.

45. The isolated polypeptide or polypeptide complex of claim 44, wherein the polypeptide that is part of the TCR-CD3 complex is human CD3ε.

46. The isolated polypeptide or polypeptide complex of claim 15, wherein A₂ comprises an antibody or antibody fragment.

47. The isolated polypeptide or polypeptide complex of claim 46, wherein the antibody or antibody fragment thereof of A₂ comprises a single chain variable fragment, a single domain antibody, Fab′, or a Fab.

48. The isolated polypeptide or polypeptide complex of claim 46, wherein the antibody or antibody fragment thereof of A₂ comprises a single chain variable fragment (scFv), a heavy chain variable domain (VH domain), a light chain variable domain (VL domain), or a variable domain (VHH) of a camelid derived single domain antibody.

49. The isolated polypeptide or polypeptide complex of claim 46, wherein the antibody or antibody fragment thereof of A₂ is humanized or human.

50. The isolated polypeptide or polypeptide complex of claim 47, wherein A₂ is the Fab or Fab′.

51. The isolated polypeptide or polypeptide complex of claim 50, wherein the Fab or Fab′ comprises (a) a Fab light chain polypeptide and (b) a Fab heavy chain polypeptide and A₁ comprises a single chain variable fragment (scFv) that comprises a scFv light chain polypeptide and a scFv heavy chain polypeptide.

52. The isolated polypeptide or polypeptide complex of claim 15, wherein the second target antigen comprises a tumor antigen.

53. The isolated polypeptide or polypeptide complex of claim 46, wherein the antibody or antibody fragment of A₂ thereof comprises an epidermal growth factor receptor (EGFR) binding domain, mesothelin binding domain, a PSMA binding domain, or a TROP2 binding domain.

54. The isolated polypeptide or polypeptide complex of claim 46, wherein the antibody or antibody fragment of A₂ comprises a PSMA binding domain or a TROP2 binding domain.

55. The isolated polypeptide or polypeptide complex of claim 13, wherein P₁ impairs binding of A₁ to the first target antigen.

56. The isolated polypeptide or polypeptide complex of claim 13, wherein P₁ is bound to A₁ through ionic interactions, electrostatic interactions, hydrophobic interactions, Pi-stacking interactions, or H-bonding interactions, or a combination thereof.

57. The isolated polypeptide or polypeptide complex of claim 13, wherein P₁ has less than 70% sequence homology to the first target antigen.

58. The isolated polypeptide or polypeptide complex of claim 15, wherein P₂ impairs binding of A₂ to the second target antigen.

59. The isolated polypeptide or polypeptide complex of claim 15, wherein P₂ is bound to A₂ through ionic interactions, electrostatic interactions, hydrophobic interactions, Pi-stacking interactions, and H-bonding interactions, or a combination thereof.

60. The isolated polypeptide or polypeptide complex of claim 15, wherein P₂ is bound to A₂ at or near an antigen binding site.

61. The isolated polypeptide or polypeptide complex of claim 15, wherein P₂ has less than 70% sequence homology to the second target antigen.

62. The isolated polypeptide or polypeptide complex of claim 15, wherein P₁ or P₂ comprises a peptide sequence of at least 10 amino acids in length.

63. The isolated polypeptide or polypeptide complex of claim 15, wherein P₁ or P₂ comprises a peptide sequence of at least 10 amino acids in length and no more than 20 amino acids in length.

64. The isolated polypeptide or polypeptide complex of claim 15, wherein P₁ or P₂ comprises a peptide sequence of at least 16 amino acids in length.

65. The isolated polypeptide or polypeptide complex of claim 15, wherein P₁ or P₂ comprises a peptide sequence of no more than 40 amino acids in length.

66. The isolated polypeptide or polypeptide complex of claim 15, wherein P₁ or P₂ comprises at least two cysteine amino acid residues.

67. The isolated polypeptide or polypeptide complex of claim 15, wherein P₁ or P₂ comprises a cyclic peptide or a linear peptide.

68. The isolated polypeptide or polypeptide complex of claim 15, wherein P₁ or P₂ comprises a cyclic peptide.

69. The isolated polypeptide or polypeptide complex of claim 15, wherein P₁ or P₂ comprises a linear peptide

70. The isolated polypeptide or polypeptide complex of claim 15, wherein L₁ is bound to a N-terminus of A₁.

71. The isolated polypeptide or polypeptide complex of claim 15, wherein L₁ is bound to a C-terminus of A₁.

72. The isolated polypeptide or polypeptide complex of claim 15, wherein L₂ is bound to a N-terminus of A₂.

73. The isolated polypeptide or polypeptide complex of claim 15, wherein L₂ is bound to a C-terminus of A₂.

74. The isolated polypeptide or polypeptide complex of claim 15, wherein L₁ or L₂ is a peptide sequence having at least 5 to no more than 50 amino acids.

75. The isolated polypeptide or polypeptide complex of claim 15, wherein L₁ or L₂ is a peptide sequence having at least 10 to no more than 30 amino acids.

76. The isolated polypeptide or polypeptide complex of claim 15, wherein L₁ or L₂ is a peptide sequence having at least 10 amino acids.

77. The isolated polypeptide or polypeptide complex of claim 15, wherein L₁ or L₂ is a peptide sequence having at least 18 amino acids.

78. The isolated polypeptide or polypeptide complex of claim 15, wherein L₁ or L₂ is a peptide sequence having at least 26 amino acids.

79. The isolated polypeptide or polypeptide complex of claim 15, wherein L₁ or L₂ has a formula comprising (G₂S)ₙ, wherein n is an integer from 1 to 3 (SEQ ID NO: 20).

80. The isolated polypeptide or polypeptide complex of claim 15, wherein L₁ has a formula selected from the group consisting of (G₂S)ₙ, (GS)ₙ, (GSGGS)ₙ (SEQ ID NO: 15), (GGGS)ₙ (SEQ ID NO: 16), (GGGGS)ₙ (SEQ ID NO: 17), and (GSSGGS)ₙ (SEQ ID NO: 18), wherein n is an integer of at least 1.

81. The isolated polypeptide or polypeptide complex of claim 13, wherein P₁ becomes unbound from A₁ when L₁ is cleaved by the tumor specific protease thereby exposing A₁ to the first target antigen.

82. The isolated polypeptide or polypeptide complex of claim 15, wherein P₂ becomes unbound from A₂ when L₂ is cleaved by the tumor specific protease thereby exposing A₂ to the second target antigen.

83. The isolated polypeptide or polypeptide complex of claim 13, wherein the tumor specific protease is selected from the group consisting of metalloprotease, serine protease, cysteine protease, threonine protease, and aspartic protease.

84. The isolated polypeptide or polypeptide complex of claim 15, wherein L₁ or L₂ comprises a urokinase cleavable amino acid sequence, a matriptase cleavable amino acid sequence, a matrix metalloprotease cleavable amino acid sequence, or a legumain cleavable amino acid sequence.

85. The isolated polypeptide or polypeptide complex of claim 15, wherein L₁ or L₂ comprises an amino acid sequence selected from the group consisting of GGGGSLSGRSDNHGSSGT (SEQ ID NO:21), GGGGSSGGSGGSGLSGRSDNHGSSGT (SEQ ID NO: 22), ASGRSDNH (SEQ ID NO: 23), LAGRSDNH (SEQ ID NO: 24), ISSGLASGRSDNH (SEQ ID NO: 25), ISSGLLAGRSDNH (SEQ ID NO: 26), LSGRSDNH (SEQ ID NO: 27), ISSGLLSGRSDNP (SEQ ID NO: 28), ISSGLLSGRSDNH (SEQ ID NO: 29), LSGRSDNHSPLGLAGS (SEQ ID NO: 30), SPLGLAGSLSGRSDNH (SEQ ID NO: 31), SPLGLSGRSDNH (SEQ ID NO: 32), LAGRSDNHSPLGLAGS (SEQ ID NO: 33), LSGRSDNHVPLSLKMG (SEQ ID NO: 34), and LSGRSDNHVPLSLSMG (SEQ ID NO: 35).

86. The isolated polypeptide or polypeptide complex of claim 15, wherein L₁ or L₂ comprises an amino acid sequence selected from the group consisting of ASGRSDNH (SEQ ID NO:23), LAGRSDNH (SEQ ID NO: 24), ISSGLASGRSDNH (SEQ ID NO: 25), and ISSGLLAGRSDNH (SEQ ID NO: 26).

87. The isolated polypeptide or polypeptide complex of claim 51, wherein the Fab light chain polypeptide of A₂ is bound to a C-terminus of the single chain variable fragment (scFv) of A₁.

88. The isolated polypeptide or polypeptide complex of claim 51, wherein the Fab heavy chain polypeptide of A₂ is bound to a C-terminus of the single chain variable fragment (scFv) of A₁.

89. The isolated polypeptide or polypeptide complex of claim 51, wherein the Fab light chain polypeptide of A₂ is bound to a N-terminus of the single chain variable fragment (scFv) of A₁.

90. The isolated polypeptide or polypeptide complex of claim 51, wherein the Fab heavy chain polypeptide of A₂ is bound to a N-terminus of the single chain variable fragment (scFv) of A₁.

91. The isolated polypeptide or polypeptide complex of claim 51, wherein the Fab heavy chain polypeptide of A₂ is bound to the scFv heavy chain polypeptide of A₁.

92. The isolated polypeptide or polypeptide complex of claim 51, wherein the Fab light chain polypeptide of A₂ is bound to the scFv heavy chain polypeptide of A₁.

93. The isolated polypeptide or polypeptide complex of claim 51, wherein the Fab heavy chain polypeptide of A₂ is bound to the scFv light chain polypeptide of A₁.

94. The isolated polypeptide or polypeptide complex of claim 51, wherein the Fab light chain polypeptide of A₂ is bound to the scFv light chain polypeptide of A₁.

95. The isolated polypeptide or polypeptide complex of claim 51, wherein the Fab heavy chain polypeptide of A₂ is bound to the scFv heavy chain polypeptide of A₁ and L₂ is bound to the Fab light chain polypeptide of A₂.

96. The isolated polypeptide or polypeptide complex of claim 51, wherein the Fab light chain polypeptide of A₂ is bound to the scFv heavy chain polypeptide of A₁ and L₂ is bound to the Fab heavy chain polypeptide of A₂.

97. The isolated polypeptide or polypeptide complex of claim 51, wherein the Fab heavy chain polypeptide of A₂ is bound to the scFv light chain polypeptide of A₁ and L₂ is bound to the Fab light chain polypeptide of A₂.

98. The isolated polypeptide or polypeptide complex of claim 51, wherein the Fab light chain polypeptide of A₂ is bound to the scFv light chain polypeptide of A₁ and L₂ is bound to the Fab heavy chain polypeptide of A₂.

99. A pharmaceutical composition comprising:
(i) the isolated polypeptide or polypeptide complex of claim 1; and
(ii) a pharmaceutically acceptable excipient.

100. An isolated recombinant nucleic acid molecule encoding the isolated polypeptide or polypeptide complex of claim 1.


SEQUENCE LISTING (40 sequences)

SEQ ID NO: 1 - protein, 8 aa, Artificial Sequence
  Description of Artificial Sequence Synthetic peptide
GSTFYTAV

SEQ ID NO: 2 - protein, 8 aa, Artificial Sequence
  Description of Artificial Sequence Synthetic peptide
IRWTALTT

SEQ ID NO: 3 - protein, 17 aa, Artificial Sequence
  Description of Artificial Sequence Synthetic peptide
AARGTLGLFTTADSYDY

SEQ ID NO: 4 - protein, 124 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSS

SEQ ID NO: 5 - protein, 17 aa, Artificial Sequence
  Description of Artificial Sequence Synthetic peptide
AARGTLGLFTTADHYDY

SEQ ID NO: 6 - protein, 124 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADHYDYWGQGTLVTVSS

SEQ ID NO: 7 - protein, 468 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 226 aa, Artificial Sequence
  Description of Artificial Sequence Synthetic polypeptide
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 226 aa, Artificial Sequence; identical to SEQ ID NO: 8
  Description of Artificial Sequence Synthetic polypeptide

SEQ ID NO: 11 - protein, 214 aa, Artificial Sequence
  Description of Artificial Sequence Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 478 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 256 aa, Artificial Sequence
  Description of Artificial Sequence Synthetic polypeptide
GGVDFCKIYSWPVCHQGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 655 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 5 aa, Artificial Sequence
  Description of Artificial Sequence Synthetic peptide
GSGGS

SEQ ID NO: 16 - protein, 4 aa, Artificial Sequence
  Description of Artificial Sequence Synthetic peptide
GGGS

SEQ ID NO: 17 - protein, 5 aa, Artificial Sequence
  Description of Artificial Sequence Synthetic peptide
GGGGS

SEQ ID NO: 18 - protein, 6 aa, Artificial Sequence
  Description of Artificial Sequence Synthetic peptide
GSSGGS

SEQ ID NO: 19 - protein, 9 aa, Artificial Sequence
  Description of Artificial Sequence Synthetic peptide
GGGGSGGGS

SEQ ID NO: 20 - protein, 9 aa, Artificial Sequence
  Description of Artificial Sequence Synthetic peptide
  SITE (1)..(9): This sequence may encompass 1-3 “Gly Gly Ser” repeating units
GGSGGSGGS

SEQ ID NO: 21 - protein, 18 aa, Artificial Sequence
  Description of Artificial Sequence Synthetic peptide
GGGGSLSGRSDNHGSSGT

SEQ ID NO: 22 - protein, 26 aa, Artificial Sequence
  Description of Artificial Sequence Synthetic peptide
GGGGSSGGSGGSGLSGRSDNHGSSGT

SEQ ID NO: 23 - protein, 8 aa, Artificial Sequence
  Description of Artificial Sequence Synthetic peptide
ASGRSDNH

SEQ ID NO: 24 - protein, 8 aa, Artificial Sequence
  Description of Artificial Sequence Synthetic peptide
LAGRSDNH

SEQ ID NO: 25 - protein, 13 aa, Artificial Sequence
  Description of Artificial Sequence Synthetic peptide
ISSGLASGRSDNH

SEQ ID NO: 26 - protein, 13 aa, Artificial Sequence
  Description of Artificial Sequence Synthetic peptide
ISSGLLAGRSDNH

SEQ ID NO: 27 - protein, 8 aa, Artificial Sequence
  Description of Artificial Sequence Synthetic peptide
LSGRSDNH

SEQ ID NO: 28 - protein, 13 aa, Artificial Sequence
  Description of Artificial Sequence Synthetic peptide
ISSGLLSGRSDNP

SEQ ID NO: 29 - protein, 13 aa, Artificial Sequence
  Description of Artificial Sequence Synthetic peptide
ISSGLLSGRSDNH

SEQ ID NO: 30 - protein, 16 aa, Artificial Sequence
  Description of Artificial Sequence Synthetic peptide
LSGRSDNHSPLGLAGS

SEQ ID NO: 31 - protein, 16 aa, Artificial Sequence
  Description of Artificial Sequence Synthetic peptide
SPLGLAGSLSGRSDNH

SEQ ID NO: 32 - protein, 12 aa, Artificial Sequence
  Description of Artificial Sequence Synthetic peptide
SPLGLSGRSDNH

SEQ ID NO: 33 - protein, 16 aa, Artificial Sequence
  Description of Artificial Sequence Synthetic peptide
LAGRSDNHSPLGLAGS

SEQ ID NO: 34 - protein, 16 aa, Artificial Sequence
  Description of Artificial Sequence Synthetic peptide
LSGRSDNHVPLSLKMG

SEQ ID NO: 35 - protein, 16 aa, Artificial Sequence
  Description of Artificial Sequence Synthetic peptide
LSGRSDNHVPLSLSMG

SEQ ID NO: 36 - protein, 6 aa, Artificial Sequence
  Description of Artificial Sequence Synthetic peptide
  SITE (1)..(6): This sequence may encompass 1-3 “Gly Ser” repeating units
GSGSGS

SEQ ID NO: 37 - protein, 15 aa, Artificial Sequence
  Description of Artificial Sequence Synthetic peptide
  SITE (1)..(15): This sequence may encompass 1-3 “Gly Ser Gly Gly Ser” repeating units
GSGGSGSGGSGSGGS

SEQ ID NO: 38 - protein, 12 aa, Artificial Sequence
  Description of Artificial Sequence Synthetic peptide
  SITE (1)..(12): This sequence may encompass 1-3 “Gly Gly Gly Ser” repeating units
GGGSGGGSGGGS

SEQ ID NO: 39 - protein, 15 aa, Artificial Sequence
  Description of Artificial Sequence Synthetic peptide
  SITE (1)..(15): This sequence may encompass 1-3 “Gly Gly Gly Gly Ser” repeating units
GGGGSGGGGSGGGGS

SEQ ID NO: 40 - protein, 18 aa, Artificial Sequence
  Description of Artificial Sequence Synthetic peptide
  SITE (1)..(18): This sequence may encompass 1-3 “Gly Ser Ser Gly Gly Ser” repeating units
GSSGGSGSSGGSGSSGGS